(19) United States
(12) Patent Application Publication
(10) Pub. No.: US 2011/0110897 A1
(43) Pub. Date: May 12, 2011

(54) Adult Human Cardiac-Derived Progenitor Cells

(76) Inventors: Richard P. Schwarz, Blue Bell, PA (US); Michael D. Schneider, London (GB); Michela Noseda, London (GB)

(21) Appl. No.: 12/812,353

(22) PCT Filed: Jan. 9, 2009
(86) PCT No.: PCT/US2009/030635

§ 371 Date: Jan. 10, 2011

Related U.S. Application Data
(60) Provisional application No. 61/010,776, filed on Jan. 11, 2008.

(51) Int. Cl. A61K 35/34; C12N 5/071; A61P 9/04; A61P 9/10

(52) U.S. Cl. 424/93.7; 435/366

(57) ABSTRACT

The present invention is based, in part, on the discovery that cardiac progenitor cells are present in and can be isolated from adult human heart. Accordingly, a cell of the present invention comprises a human adult cardiac-derived progenitor cell capable of differentiating into a cardiac myocyte where said cell is isolated according to the expression of specific biomarkers, identified elsewhere herein. The present invention also includes methods of use of an adult cardiac-derived progenitor cell in the treatment of heart disease.

### BACKGROUND OF THE INVENTION

Heart failure is a severe deficiency in ventricular pump function and arises through a number of causes. These causes may be factors intrinsic to a cardiac muscle cell's contractility, such as altered expression or operation of calcium-cycling proteins, components of the sarcomere, and enzymes for cardiac energy production; or factors extrinsic to cardiac muscle cells, such as interstitial fibrosis and myocyte loss unmatched by myocyte replacement.

Like the brain, the heart has long been considered a post-mitotic organ. Cardiac myocytes have traditionally been regarded as terminally differentiated cells that adapt to increased work and compensate for cell loss due to injury or disease exclusively through cell hypertrophy. Because of this long-standing view, treatment of heart disease has of necessity, focused on minimizing initial cardiac myocyte loss and maximizing recovery of “hibernating” cardiac myocytes following an acute myocardial infarction or ischemic event by rapid restoration of tissue perfusion. Subsequent clinical management focuses on efforts to prevent or manage deleterious ventricular remodeling and hypertrophy. More aggressive interventions such as cardiac transplantation and the use of left ventricular (LV) assist devices (either as a bridge to transplantation or as destination therapies) are thwarted by significant comorbidity and/or fmite effectiveness. Thus, conventional palliative medical management does not correct, or even attempt to correct, the fundamental underlying defect in cardiac muscle cell number that occurs as a result of cardiac myocyte death.

To date, several attempts have been made to use stem cells from various sources to repair damaged or failing myocardial tissue. Autologous transplantation of skeletal myoblasts was thought to be particularly promising because of the cells’ highly proliferative nature and commitment to a well-differentiated myogenic lineage. However, in clinical trials, although these cells became well integrated into the host heart, they failed to electrically couple with host cardiomyocytes, causing potentially life-threatening arrhythmias. Autologous transplantation of endogenous hematopoietic cells has resulted in only rare cardiogenic conversion. The predominant in vivo effect of bone marrow or endothelial progenitor cell transplantation has been neoangiogenesis, together possibly with paracrine effects on myocyte survival, function, and endogenous stem cell recruitment, but not cardiac myogenesis itself.

Recently, evidence of cardiomyocyte proliferation in damaged human heart implies that cardiac regeneration may occur by resident or extra-cardiac stem cells. The origin and specification of these stem cells remains unknown.

It has been found that the adult heart of a mouse, like other adult organs, contains a population of cells denominated side population (SP) because of their ability to efflux metabolic markers such as rhodamine and Hoechst 33342 dye. SP cells of adult murine cardiac cells express the surface label stem cell antigen-1 (Sca-1), an orphan receptor of the urokinase plasminogen activator receptor superfamily. Mouse cardiac Sca-1 positive (Sea-1^{pos}) cells were negative for hematopoietic cell surface and transcriptional markers. These cells were also enriched for telomerase, lacked transcripts for cardiac structural genes, but expressed many of the known cardiogenic (heart forming) transcription factors such as Gata4, Mef2c, and Tbx5. Together, the absence of hematopoietic transcription factors and the presence of cardiogenic transcription factors determine that the murine cardiac Sca-1^{pos} cells are fundamentally distinct from hematopoietic cells at the molecular level and that their properties consequently cannot be predicted or inferred from work on hematopoietic cells.

Sca-1^{pos} cells administered to mice subjected to myocardial ischemia/reperfusion injury homed to the site of injury, constituting 15-20% of the infarct border zone. Two weeks later, all donor Sca-1^{pos} cells expressed sarcomeric actin, cardiac troponin, and the gap junction protein connexin 43. While mouse cardiac Sca-1^{pos} cells remain undifferentiated in routine culture, they can be induced to differentiate using 5′-azacytidine, oxytocin, and growth factors.

Despite recent findings in mice, no authentic ortholog of Sca-1 exists in the human genome and regenerating fully functioning mature cardiomyocytes from autologous, adult, human, cardiac stem/progenitor cells remains a distant goal. The present invention achieves this goal.

### SUMMARY OF THE INVENTION

In one embodiment, the present invention comprises an isolated human adult progenitor cell, wherein the cell is isolated from a cardiac tissue sample, wherein the cell expresses an Abcg2 protein or an mRNA encoding Abcg2, wherein the cell also expresses at least one cardiac transcription factor protein selected from the group consisting of dHAND, Gata4, Mef2a, Tbx2, Tbx5, Tbx20, or an mRNA encoding at least one of the cardiac transcription factor, and further wherein, the cell does not substantially express a c-kit protein or an mRNA encoding c-kit at the time of cell isolation. In one aspect, the cell further expresses a protein or a nucleic acid encoding a protein selected from the group of mesenchymal stem cell markers consisting of CD105, CD90, CD44. In another aspect, the cell does not express at least one hematopoietic biomarker selected from the group consisting of CD45, CD34, CD31, Lin, Lmo2, Gata2, and Tal. In yet another aspect, the cell will differentiate into a cardiac muscle cell.

Another embodiment of the present invention comprises a pharmaceutical composition comprising a therapeutically effective amount of an isolated human adult progenitor cell, wherein the cell is isolated from a cardiac tissue sample, wherein the cell expresses an Abcg2 protein or an mRNA encoding Abcg2, wherein the cell also expresses at least one cardiac transcription factor protein selected from the group consisting of dHAND, Gata4, Mef2a, Tbx2, Tbx5, Tbx20, or an mRNA encoding at least one the cardiac transcription factor, and further wherein, the cell does not substantially express a c-kit protein or an mRNA encoding c-kit at the time of cell isolation. In one aspect, the pharmaceutical composition comprises a cell, wherein the cell also expresses a protein or a nucleic acid encoding a protein selected from the group of mesenchymal stem cell markers consisting of CD105, CD90, CD44. In still another aspect, the cell does not express at least one hematopoietic biomarker selected from the group consisting of CD45, CD34, CD31, Lin, Lmo2, Gata2, and Tal. In yet another aspect, the cell will differentiate into a cardiac muscle cell.

Yet another embodiment of the invention comprises a cultured human adult progenitor cell, wherein the cell is isolated from a cardiac tissue sample, wherein the cell expresses an Abcg2 protein or an mRNA encoding Abcg2, wherein the cell also expresses at least one cardiac transcription factor protein selected from the group consisting of dHAND, Gata4, Mef2a, Tbx2, Tbx5, Tbx20, or an mRNA encoding at least one the cardiac transcription factor, and further wherein, the cell does not substantially express a c-kit protein or an mRNA encoding c-kit at the time of cell isolation. In one aspect, the cell further expresses a protein or a nucleic acid encoding a protein selected from the group of mesenchymal stem cell markers consisting of CD105, CD90, CD44. In another aspect, the cell does not express hematopoietic biomarkers selected from the group consisting of CD45, CD34, Lmo2, Gata2, and Tal. In still another aspect, the cell will differentiate into a cardiac muscle cell.

Still another embodiment of the present invention comprises a pharmaceutical composition comprising a therapeutic amount of a cultured human adult progenitor cell, wherein the cell is isolated from a cardiac tissue sample, wherein said cell expresses an Abcg2 protein or an mRNA encoding Abcg2, wherein the cell also expresses at least one cardiac transcription factor protein selected from the group consisting of dHAND, Gata4, Mef2a, Tbx2, Tbx5, Tbx20, or an mRNA encoding at least one the cardiac transcription factor, and further wherein, the cell does not substantially express a c-kit protein or an mRNA encoding c-kit at the time of cell isolation. In one aspect, the cell further expresses a protein or a nucleic acid encoding a protein selected from the group of mesenchymal stem cell markers consisting of CD105, CD90, CD44. In another aspect, the cell does not express at least one hematopoietic biomarker selected from the group consisting of CD45, CD34, CD31, Lin, Lmo2, Gata2, and Tal. In still another aspect, the cell will differentiate into a cardiac muscle cell.

Yet another embodiment of the present invention comprises a substantially pure population of adult human progenitor cells, wherein the population of cells is derived from a cardiac tissue sample obtained from a human, wherein the population of cells of the population is selected by fluorescence or magnetic cell sorting, and wherein the population of cells expresses an Abcg2 protein or an mRNA encoding Abcg2, wherein the cell also expresses at least one cardiac transcription factor protein selected from the group consisting of dHAND, Gata4, Mef2a, Tbx2, Tbx5, Tbx20, or an mRNA encoding at least one the cardiac transcription factor, and further wherein, the population of cells does not substantially express a c-kit protein or an mRNA encoding c-kit at the time of cell selection. In one aspect, the population of cells expresses mesenchymal stem cell markers selected from the list consisting of CD 105, CD90, CD44. In another aspect, the population of cells does not express at least one hematopoietic biomarker selected from the group consisting of CD45, CD34, CD31, Lin, Lmo2, Gata2, and Tal. In yet another aspect, the population of cells will differentiate into cardiac muscle cells.

Another embodiment of the present invention comprises a composition comprising a therapeutic amount of an isolated adult human progenitor cell, wherein the cell is derived from a cardiac tissue sample obtained from a human, wherein the cell expresses an Abcg2 protein or an mRNA encoding Abcg2, wherein the cell also expresses at least one cardiac transcription factor protein selected from the group consisting of dHAND, Gata4, Mef2a, Tbx2, Tbx5, Tbx20, or an mRNA encoding at least one the cardiac transcription factor, and further wherein, the cell does not substantially express a c-kit protein or an mRNA encoding c-kit at the time of cell isolation. In one aspect, the composition further comprises a pharmaceutical acceptable carrier. In another aspect, the cell further expresses a protein or a nucleic acid encoding a protein selected from the group of mesenchymal stem cell markers consisting of CD105, CD90, CD44. In still another aspect, the cell does not express at least one hematopoietic biomarker selected from the group consisting of CD45, CD34, CD31, Lin, Lmo2, Gata2, and Tal. In another aspect, the cell will differentiate into a cardiac muscle cell.

Still another embodiment of the present invention comprises a method of treating a patient with heart disease, the method comprising the steps of administering to the patient a therapeutically effective amount of a human progenitor cell, wherein the cell is isolated from a cardiac tissue sample, wherein when the cell is isolated, the cell expresses an Abcg2 protein or an mRNA encoding Abcg2, wherein the cell also expresses at least one cardiac transcription factor protein selected from the group consisting of dHAND, Gata4, Mef2a, Tbx2, Tbx5, Tbx20, or an mRNA encoding at least one the cardiac transcription factor, and further wherein, the cell does not substantially express a c-kit protein or an mRNA encoding c-kit at the time of cell isolation. In one aspect, the cell further expresses a protein or a nucleic acid encoding a protein selected from the group of mesenchymal stem cell markers consisting of CD105, CD90, CD44. In another aspect, the cell does not express at least one hematopoietic biomarker selected from the group consisting of CD45, CD34, CD31, Lin, Lmo2, Gata2, and Tal. In yet another aspect, the cardiovascular disease is selected from the list consisting of coronary artery disease, myocardial infarction, ischemic heart disease, and heart failure. In still another aspect, the cell is admixed with a pharmaceutical acceptable carrier. In another aspect, the cell is autologous.

Yet another embodiment of the present invention comprises a method of treating damaged myocardium in a patient, the method comprising the steps of: (a) obtaining cells from an adult cardiac tissue sample; (b) selecting cells one or more times, before or after expanding the cells, to generate a substantially pure population of cardiac progenitor cells that are c-kit^{neg} and Abcg2^{pos} at the time of cell selection; (c) expanding the cells to form a substantially pure population of adult human cardiac progenitor cells; (d) administering a therapeutically effective amount of the cardiac progenitor cells to the patient.

Still another embodiment of the present invention comprises a method of obtaining a population of human cardiac progenitor cells, the method comprising the steps of: (a) obtaining cells from a cardiac tissue sample; (b) selecting cells one or more times, before or after expanding the cells, using either fluorescent or magnetic cell sorting techniques, to generate a substantially pure population of cardiac progenitor cells, wherein the cells are substantially c-kit^{neg} and Abcg2^{pos} at the time of sorting; (c) expanding the cells to form a substantially pure population of adult human cardiac progenitor cells. In one aspect, the cells are sorted at least once using one or more biomarker.

### BRIEF DESCRIPTION OF THE DRAWINGS

For the purpose of illustrating the invention, there are depicted in the drawings certain embodiments of the invention. However, the invention is not limited to the precise arrangements and instrumentalities of the embodiments depicted in the drawings.

FIG. 1, comprising FIG. 1A and FIG. 1B, is a series of charts comparing two methods of isolating human adult cardiac derived progenitor cells. FIG. 1A is a graph depicting the number of cells at the end of passage number 1(P1) per gram of starting tissue obtained from tissue that was isolated using either the explant or cell dissociation method. FIG. 1B is a graph depicting the number of days required for cells obtained using either the explant or dissociation method to reach Passage 1.

FIG. 2, comprising FIG. 2A through FIG. 2C, is a series of charts depicting propagation of human cardiac-derived cells using collagen-coated or poly-D lysine coated dishes. FIG. 2A is a graph depicting growth curves for cells grown under different conditions. FIG. 2B depicts flow cytometry analysis of cells stained with BrdU and propidium iodide (PI). Graphs show distribution of cells in the various phases of the cell cycle. G0/G1 characterized by low BrdU and PI, S-phase with high levels of BrdU which indicate active DNA synthesis and G2/M with low BrdU and double amount of PI (because of DNA duplication). FIG. 2C is a graph depicting the per cent (%) cells in S -phase (actively proliferating) of the cell cycle in each condition. C=collagen; D=poly-D lysine.

FIG. 3, comprising FIG. 3A and FIG. 3B, is a series of photographs depicting the characterization of human derived cardiac cells obtained using a dissociation method. RNA obtained from cells was probed for a variety of potential biomarkers. Tissue was obtained from the left ventricle (LV). ULA=ultra low attachment; C=collagen; D=poly-D lysine; Heart; PBMC=peripheral blood mononuclear cells. Negative controls were run without DNA template.

FIG. 4, comprising FIG. 4A through FIG. 4D, is a series of charts characterizing side population (SP) cells present in human cardiac-derived cultures. FIG. 4A is a series of graphs depicting human cardiac-derived cells analyzed by flow cytometry according to their ability to efflux Hoechst dye 33342, thereby identifying side population (SP) cells and non-SP (NSP) cells. As a control, cells were treated with Verapamil (inhibitor of Hoechst extrusion) showing the expected disappearance of the SP cells. FIG. 4B is a series of graphs depicting cells gated on the SP population (top panel) or NSP (bottom panel). Results show that SP cells from human cardiac-derived cells are substantially negative for expression of hematopoietic progenitor marker CD45 and for antigens recognized by the hematopoietic lineage marker cocktail (Lin). NSP cells exhibit low levels of Lin+ cells. FIG. 4C is a graph depicting the % of samples positive for SP cells when human cardiac-derived cells were grown on either collagen (C) or poly-D lysine (D). FIG. 4D is a graph depicting the % SP cells—when human cardiac-derived cells were grown on either collagen (C) or poly-D lysine (D).

FIG. 5, comprising FIG. 5A through FIG. 5E, is a series of images depicting the identification of SP cells in human cardiac-derived cultures using different types of inhibitors. FIG. 5A through FIG. 5C depict two-color flow cytometry indicating human cardiac SP cells (region encompassed by solid polygon), by Hoechst dye exclusion. FIG. 5A depicts Hoechst dye exclusion in untreated SP cells. FIG. 5B depicts loss of Hoechst dye exclusion in cells treated with the pump inhibitor Reserpine (Res). FIG. 5C depicts loss of Hoechst dye exclusion in cells treated with another pump inhibitor, Fumitremorgin C (FTC). FIGS. 5D and 5E are SP cells sorted by flow cytometry and subsequently maintained in culture.

FIG. 6, comprising FIG. 6A through FIG. 6D, is a series of charts depicting expression of extrusion pumps as putative molecular determinants of cells with the cardiac SP phenotype. FIG. 6A is a graph depicting the % of samples positive for Abcg2 which were grown on collagen or poly-D lysine. FIG. 6B depicts Abcg2 expression in terms of % of cells positive for Abcg2 in cultured cells grown on collagen or poly-D lysine. FIG. 6C is a graph depicting the % of samples positive for MDR1 which were grown on collagen or poly-D lysine. FIG. 6D depicts MDR1 expression in terms of % of cells positive for MDR1 in cells grown on collagen or poly-D lysine.

FIG. 7, comprising FIG. 7A through FIG. 7C, is a series of graphs depicting the expression of extrusion pumps as (Abcg2 and MDR1) as putative molecular determinants of the cardiac SP phenotype. Two-color flow cytometry was used to determine the percentage of cells that were Abcg2+/MDR1−; Abcg2−/MDR1+; and Abcg2+/MDR1+. FIG. 7A depicts the separation of MDR1+ cells (16.98%). Isotype-specific irrelevant control antibodies (IgG-PE) were used in lieu of Abcg2. FIG. 7B depicts the separation of Abcg2+ cells (0.07%). Isotype-specific irrelevant control antibodies (IgG-FITC) were used in lieu of MDR1. FIG. 7C depicts the separation of Abcg2+/MDR+ SP cells (0.1%) and Abcg2−/MDR1+ SP ells (15.01%) (no Abcg2+/MDR1− cells were identified).

FIG. 8 is a drawing depicting the characterization of human cardiac-derived cells using mesenchymal stem-cell markers to define sub-populations of cardiac progenitor cells. CD31−, but not CD31+ cardiac SP cells exhibit functional cardiomyogenic differentiation (Pfister et al., 2005, Circ. Res). CD31 can be co-expressed with CD 105 and CD44 on endothelial cells. Also, mesenchymal stem cells from the hematopoietic system and other organs are by definition CD31−.

FIG. 9, comprising FIG. 9A through FIG. 9F, is a series of graphs depicting the results of three-color flow cytometry performed on human derived cardiac cells to determine expression of mesenchymal markers CD44, CD90, and CD105. FIG. 9A and

FIG. 9D are histograms depicting expression of CD44 (grey). Black histogram is the profile of the isotype control. FIG. 9B is a dot blot gated on CD44− populations to show isotype controls. FIG. 9C is a dot blot gated on CD44+ populations to show isotype controls. FIG. 9E is a dot blot gated on CD44− populations to show CD90/CD105 staining. FIG. 9F is a dot blot gated on CD44+ populations to show CD90/CD105 staining.

FIG. 10, comprising FIG. 10A through FIG. 10F, is a series of graphs depicting the results of three-color flow cytometry performed on human derived cardiac cells to determine expression of CD31 and mesenchymal markers CD90/CD105. FIG. 10A and FIG. 10D are histograms depicting expression of CD31 (grey). Black histogram is the profile of the isotype control. FIG. 10B is a dot blot gated on CD31− populations to show isotype controls. FIG. 10C is a dot blot gated on CD31+ populations to show isotype controls. FIG. 10E is a dot blot gated on CD31− populations to show CD90/CD105 staining. FIG. 10F is a dot blot gated on CD31+ populations to show CD90/CD105 staining.

FIG. 11, comprising FIG. 11A through FIG. 11F, is a series of graphs depicting the results of three-color flow cytometry performed on human derived cardiac cells to determine expression of mesenchymal markers CD31 and CD44/CD105. FIG. 11A and FIG. 11D are histograms depicting expression of CD31 (grey). Black histogram is the profile of the isotype control. FIG. 11B is a dot blot gated on CD31− populations to show isotype controls. FIG. 11C is a dot blot gated on CD31+ populations to show isotype controls. FIG. 11E is a dot blot gated on CD31− populations to show CD44/CD105 staining. FIG. 11F is a dot blot gated on CD31+ populations to show CD44/CD105 staining.

FIG. 12 is a graph depicting the decline in ejection fraction as measured by echocardiograms. All echocardiograms measured parameters of cardiac function on three mice pre- and post-infarction that were injected with Abcg2+ human cardiac derived progenitor cells, two mice that were injected with Abcg2− human cardiac derived progenitor cells, or two control mice injected with PBS.

FIG. 13 is a graph depicting the percent decrease of ejection fraction as measured by echocardiograms. All echocardiograms measured parameters of cardiac function on three mice pre- and post-infarction that were injected with Abcg2+ human cardiac derived progenitor cells, two mice that were injected with Abcg2− human cardiac derived progenitor cells, or two control mice injected with PBS.

FIG. 14 is a graph depicting functional shortening in the myocardium following infarction as measured by echocardiograms. All echocardiograms measured parameters of cardiac function on three mice pre- and post-infarction that were injected with Abcg2+ human cardiac derived progenitor cells, two mice that were injected with Abcg2− human cardiac derived progenitor cells, or two control mice injected with PBS.

FIG. 15, comprising FIG. 15A and FIG. 15B, are graphs depicting left ventricular (LV) dimensional diastole (LVIDd) and systole (LVIDS) before and after induced myocardial infarction in three mice that were injected with Abcg2+ human cardiac derived progenitor cells, two mice that were injected with Abcg2− human cardiac derived progenitor cells, or two control mice injected with PBS.

FIG. 16, comprising FIG. 16A through FIG. 16D, is a series of images depicting ST elevation in the electrocardiogram (ECG) of mice measured after ligation of the left anterior descending coronary artery (LAD). Data from two mice (mouse 440 and mouse 443) are shown, both of which were injected with Abcg2+ human cardiac derived progenitor cells post infarction.

FIG. 17, comprising FIG. 17A and FIG. 17B, is a series of images depicting ST elevation in the electrocardiogram (ECG) of a mouse measured after ligation of the left anterior descending coronary artery (LAD). Data from mouse 445 are shown, which was injected with Abcg2− human cardiac derived progenitor cells post infarction.

FIG. 18, comprising FIG. 18A and FIG. 18B, is a series of images depicting ST elevation in the electrocardiogram (ECG) of a mouse measured after ligation of the left anterior descending coronary artery (LAD). Data from mouse 448 are shown, which was injected PBS as a control.

### DETAILED DESCRIPTION OF THE INVENTION

The present invention is based, in part, on the discovery that cardiac progenitor cells are present in and can be isolated from adult human heart. Accordingly, a cell of the present invention comprises a human adult cardiac-derived progenitor cell capable of differentiating into a cardiac myocyte where said cell is characterized according to the expression of specific biomarkers identified elsewhere herein.

The methods of the present invention include isolating, culturing, expanding, and selecting an adult cardiac-derived progenitor cell.

In another embodiment, the present invention includes compositions and methods useful for obtaining a substantially pure population of human adult cardiac-derived progenitor cells. In yet another embodiment, the present invention includes a method of repairing damaged or diseased myocardial tissue in a human by administering an adult cardiac-derived progenitor cell to a human with heart disease. In yet another embodiment, the present invention provides compositions and methods for restoring or regenerating cardiac myocytes or tissue lost due to disease or damage in a human by administering an adult cardiac-derived progenitor cell to a human with heart disease. In still another embodiment, the present invention includes a method of treating heart disease by administering an adult cardiac-derived progenitor cell to an individual with heart disease.

### Definitions:

As used herein, each of the following terms has the meaning associated with it in this section.

The articles “a” and “an” are used herein to refer to one or to more than one (i.e. to at least one) of the grammatical object of the article. By way of example, “an element” means one element or more than one element.

The term “about” will be understood by persons of ordinary skill in the art and will vary to some extent on the context in which it is used.

The term “sample,” or “cardiac sample,” as used herein, refers to tissue obtained from a heart. The tissue may be procured in any way that preserves the viability of the cells of the invention. Non-limiting examples of sources a sample may be procured from include a biopsy, a human organ donor, an organ removed prior to transplantation, and a surgical sample.

The term “autogeneic,” “autologous,” or, “self,” as used herein, indicates the origin of a cell with respect to a recipient of the cell. Thus, a cell is autogeneic if the cell was derived from an individual (the “donor”) or a genetically identical individual and is to be readministered to the same individual. An autogeneic cell can also be a progeny of an autogeneic cell. The term also indicates that cells of different cell types are derived from the same donor or genetically identical donors. Thus, an effector cell and an antigen presenting cell are said to be autogeneic if they were derived from the same donor or from an individual genetically identical to the donor, or if they are progeny of cells derived from the same donor or from an individual genetically identical to the donor.

Similarly, the term “allogeneic,” or “non-self,” as used herein, indicates the origin of a cell with respect to a recipient of the cell. Thus, a cell and progeny thereof is allogeneic if the cell was derived from an individual not genetically identical to the recipient to whom it is to be administered; in particular, the term relates to non-identity in expressed MHC molecules. The term also indicates that cells of different cell types are derived from genetically non-identical donors, or if they are progeny of cells derived from genetically non-identical donors. For example, an APC is said to be allogeneic to an effector cell if they are derived from genetically non-identical donors.

As used herein, “syngeneic” refers to biological material derived from a genetically-identical individual (e.g. identical twin) as the individual into whom the material will be introduced.

By “complementary” when used to refer to a biomarker herein, is intended that detection of the combination of biomarkers on the surface of a cell results in the successful identification of adult cardiac-derived progenitor cell in a greater percentage of cases than would be identified if only one of the biomarkers was used. Thus, in some cases, a more accurate characterization of an adult cardiac-derived progenitor cell can be made by using at least two biomarkers.

The term “stem cell,” as used herein, refers to a pluripotent or lineage-uncommitted cell which is potentially capable of an unlimited number of mitotic divisions to either renew itself or to produce progeny cells, and which is unambiguously able to differentiate into multiple cell types derived from single-cell clones.

The term, “progenitor cells” as used herein, refers to a lineage-committed cell considered to be incapable of giving rise to numerous cell types that phenotypically differ from each other. Instead, progenitor cells give rise to one or possibly two lineage-committed cell types.

As used herein, the term “multipotential” or “multipotentiality” is meant to refer to the capability of a stem cell to differentiate into more than one type of cell.

As used herein, to “treat” means reducing the frequency with which symptoms of a disease, defect, disorder, or adverse condition, and the like, are experienced by a patient.

As used herein, a “therapeutically effective amount” is the amount of a composition of the invention sufficient to provide a beneficial effect to the individual to whom the composition is administered. For instance, with regard to the administration of cells to an individual, “therapeutically effective amount” is the amount of cells which is sufficient to provide a beneficial effect to the subject to which the cells are administered.

A “therapeutic” treatment is a treatment administered to a subject who exhibits at least one symptom of pathology for the purpose of treating or alleviating the at least one symptom.

As used herein, the term “growth medium” is meant to refer to a culture medium that promotes proliferation of cells. A growth medium will generally contain animal serum. In some instances, the growth medium may not contain animal serum.

“Differentiation medium” is used herein to refer to a growth medium comprising an additive or a lack of an additive such that a stem cell, a progenitor cell, a cardiac-derived adult stem cell or other such progenitor cell, that is not fully differentiated when incubated in the medium, develops into a cell with some or all of the characteristics of a differentiated cell.

As used herein, a “growth factor” is a substance that stimulates proliferation, division and/or maturation of a cell. A growth factor, as used herein, encompasses a “cytokine” or a “chemokine.” Non-limiting examples of growth factors useful in the instant invention include erythropoietin (EPO), macrophage colony stimulating factor, thrombopoietin (TPO), growth hormone (GH), interleukin 1-α and 1-β, interleukin 3 (IL-3), interleukin 4 (IL-4), interleukin 5 (IL-5), interleukin 6 (IL-6), interleukin 7 (IL-7), interleukin 9 (IL-9), interleukin 10 (IL-10), interleukin 11 (IL-11), interleukin 13 (IL-13), c-kit ligand/stem cell factor (SCF), insulin, insulin-like growth factors, such as IGF-2, epidermal growth factor (EGF), transforming growth factor beta, such as activin, fibroblast growth factor (FGF), such as FGF-1, FLT-3/FLK-2 ligand, granulocyte-macrophage colony-stimulating factor (GM-CSF), granulocyte colony-stimulating factor (G-CSF), and macrophage colony stimulating factor (M-CSF). Growth factors are typically used at concentrations of between picogram/ml to milligram/ml levels.

An “isolated cell” refers to a cell which has been separated from other components and/or cells which naturally accompany the cell in a tissue or mammal.

As used herein, a “substantially purified cell” is a cell that has been purified from other cell types with which it is normally associated in its naturally-occurring state.

“Expandability” is used herein to refer to the capacity of a cell to proliferate, for example, to expand in number or, in the case of a population of cells, to undergo population doublings. Cells are expanded in culture when they are placed in a growth medium under conditions that facilitate cell growth and/or division, resulting in a larger population of the cells.

“Proliferation” is used herein to refer to the reproduction or multiplication of similar forms, especially of cells. That is, proliferation encompasses production of a greater number of cells, and can be measured by, among other things, simply counting the numbers of cells, measuring incorporation of ³H-thymidine into the cell, and the like. The rate of cell proliferation is typically measured by the amount of time required for the cells to double in number, otherwise known as the doubling time.

As used herein, “cell culture” refers to the process whereby cells, taken from a living organism, are grown under controlled conditions.

A “primary cell culture” refers to a culture of cells, tissues or organs taken directly from an organism and before the first subculture.

As used herein, “subculture” refers to the transfer of cells from one growth container to another growth container.

As used herein, a “passage” refers to a round of subculturing. Thus, when cells are subcultured, they are referred to as having been passaged. A specific population of cells, or a cell line, is sometimes referred to or characterized by the number of times it has been passaged. For example, a cultured cell population that has been passaged ten times may be referred to as a P10 culture. The primary culture, i.e., the first culture following the isolation of cells from tissue, is designated P0. Following the first subculture, the cells are described as a secondary culture (P1 or passage 1). After the second subculture, the cells become a tertiary culture (P2 or passage 2), and so on. It will be understood by those of skill in the art that there may be many population doublings during the period of passaging; therefore the number of population doublings of a culture is greater than the passage number. The expansion of cells (i.e., the number of population doublings) during the period between passaging depends on many factors, including but, not limited to, the seeding density, substrate, medium, and time between passaging.

“Exogenous” refers to any material introduced into or produced outside an organism, cell, or system.

As used herein, the term “phenotype” or “phenotypic characteristics” should be construed to mean the expression of a specific biomarker protein or nucleic acid, or a combination of biomarker proteins or nucleic acids, that distinguishes one cell from another.

“Encoding” refers to the inherent property of specific sequences of nucleotides in a polynucleotide, such as a gene, a cDNA, or an mRNA, to serve as templates for synthesis of other polymers and macromolecules in biological processes having either a defined sequence of nucleotides (i.e., rRNA, tRNA and mRNA) or a defined sequence of amino acids and the biological properties resulting therefrom. Thus, a gene encodes a protein if transcription and translation of mRNA corresponding to that gene produces the protein in a cell or other biological system. Both the coding strand, the nucleotide sequence of which is identical to the mRNA sequence and is usually provided in sequence listings, and the non-coding strand, used as the template for transcription of a gene or cDNA, can be referred to as encoding the protein or other product of that gene or cDNA.

Unless otherwise specified, a “nucleotide sequence encoding an amino acid sequence” includes all nucleotide sequences that are degenerate versions of each other and that encode the same amino acid sequence. Nucleotide sequences that encode proteins and RNA may include introns.

An “isolated nucleic acid” refers to a nucleic acid segment or fragment which has been separated from sequences which flank it in a naturally occurring state, i.e., a DNA fragment which has been removed from the sequences which are normally adjacent to the fragment, i.e., the sequences adjacent to the fragment in a genome in which it naturally occurs. The term also applies to nucleic acids which have been substantially purified from other components which naturally accompany the nucleic acid, i.e., RNA or DNA or proteins, which naturally accompany it in the cell. The term therefore includes, for example, a recombinant DNA which is incorporated into a vector, into an autonomously replicating plasmid or virus, or into the genomic DNA of a prokaryote or eukaryote, or which exists as a separate molecule (i.e., as a cDNA or a genomic or cDNA fragment produced by PCR or restriction enzyme digestion) independent of other sequences. It also includes a recombinant DNA which is part of a hybrid gene encoding additional polypeptide sequence.

In the context of the present invention, the following abbreviations for the commonly occurring nucleic acid bases are used. “A” refers to adenosine, “C” refers to cytosine, “G” refers to guanosine, “T” refers to thymidine, and “U” refers to uridine.

“Recombinant polynucleotide” refers to a polynucleotide having sequences that are not naturally joined together. An amplified or assembled recombinant polynucleotide may be included in a suitable vector, and the vector can be used to transform a suitable host cell. A recombinant polynucleotide may serve a non-coding function (e.g., promoter, origin of replication, ribosome-binding site, etc.) as well.

By “pharmaceutically acceptable carrier” is meant any carrier, diluent or excipient which is compatible with the biological component of a pharmaceutical composition and not deleterious to the recipient.

### Description:

In the present invention it has been discovered that the adult human heart comprises a population of progenitor cells capable of self-renewal and replication. This disclosure describes a novel, substantially pure population of cardiac-derived progenitor cells that can be obtained from the adult myocardium, can be expanded, and can be differentiated into cardiomyocytes. A cell of the invention, namely, an adult human cardiac-derived progenitor cell, is characterized according to the expression of certain biomarkers disclosed herein. Cells of the invention can be isolated to form a substantially pure population of cells.

A “substantially pure population” of cells means that at least about 70% (e.g., about 75%, 80%, 85%_{,} 90%, 95%, 99% or 100%, or any whole integer encompassed therein) of the cells present (e.g., not including feeder layer cells, if present) are adult cardiac-derived progenitor cells as described herein or cells that have differentiated therefrom. Methods are provided herein for further increasing the number of adult cardiac-derived progenitor cells in a population and for the use of these cells as therapeutic agents.

### I. Compositions

A. Adult Human Cardiac-Derived Progenitor Cells

A cell of the present invention is a human adult cardiac-derived progenitor cell. In one embodiment, a cell of the present invention is derived from a side population (SP) of adult cardiac-derived progenitor cells.

The adult cardiac-derived progenitor cells described herein differ from other previously described adult- or otherwise-derived cardiac stem- or progenitor cells in several ways. First, the cell of the present invention can be derived from samples obtained from an adult human donor. Second, the cell of the present invention is characterized according to the expression of specific cell surface markers including ATP-binding cassette (ABC) transporters such as Abcg2 and MDR1; mesenchymal stem cell markers, such as CD105, CD90, CD44; and cardiogenic transcription factors, such as dHAND, GATA4, MEF2A, Tbx2, Tbx5, Tbx20. Third, the cell of the present invention does not express c-kit at the time of characterization. Fourth, a cell of the present invention does not express hematopoietic transcription factors at the time of characterization. Accordingly, an adult cardiac-derived progenitor cell of the invention, is a cell characterized according to the expression of at least one of the following: a detectable amount of Abcg2 protein or an mRNA encoding Abcg2 (Abcg2^{pos}); a detectable amount of MDR1 protein or an mRNA encoding MDR1 (MDR1^{pos}); a detectable amount of CD105 protein or an mRNA encoding CD105 (CD105^{pos}); a detectable amount of CD90 protein or an mRNA encoding CD90 (CD90^{pos}); a detectable amount of CD44 protein or an mRNA encoding CD44 (CD44^{pos}).

An adult cardiac-derived progenitor cell of the invention is further characterized because it does not express at least one of the following: a detectable amount of c-kit protein or an mRNA encoding c-kit (c-kitneg); a detectable amount of Lmo2 protein or an mRNA encoding Lmo2 (Lmo^{neg}); a detectable amount of GATA2 or an mRNA encoding GATA2 (GATA2^{neg}); a detectable amount of Tal or an mRNA encoding Tal (Tal^{neg}); a detectable amount of CD45 or an mRNA encoding CD45 (CD45^{neg}); a detectable amount of Lin cocktail or an mRNA encoding Lin (Lin^{neg}).

The cells described herein are capable of cardiac repair. In one embodiment, the cells of the invention are capable of myogenesis. In another embodiment, the cells of the invention are capable of angiogenesis. Accordingly, the cells of the present invention can be used to treat cardiac tissue damaged due to injury or disease. It is understood by those of skill in the art that the term treating, as used herein, includes repairing, replacing, augmenting, improving, rescuing, repopulating, or regenerating.

It may be desirable to induce differentiation of the cells described herein in a controlled manner and/or by employing factors which are not easily or desirably introduced into the damaged cardiac tissue. Therefore, in one embodiment of the invention, the cells described herein can be induced to differentiate prior to being introduced into the recipient by, for example, in vitro exposure to extracellular and/or intracellular factors such as trophic factors, cytokines, mitogens, hormones, cognate receptors for the foregoing, and the like. In another embodiment, the cells of the invention are not induced to differentiate, but are introduced into the recipient as a substantially pure population of adult cardiac-derived progenitor cells that may differentiate following introduction into the recipient.

In one embodiment of the present invention, the adult cardiac-derived progenitor cell of the present invention is autologous. That is, a cell of the invention is procured from a donor and returned to the same individual after selection and expansion of said cell; i.e. donor and recipient are the same individual. In another embodiment of the present invention, an adult cardiac-derived progenitor cell of the present invention is allogenic. That is, the cell of the invention is procured from a donor but administered to a different individual after selection and expansion of said cell; i.e. the donor and recipient are genetically different individuals.

In another embodiment of the invention, the cells procured from a donor, whether from the heart or from other sources, lack one or more of the mRNAs required for the definition of an adult cardiac-derived progenitor cell. Modification of such a donor cell to resemble an authentic adult cardiac-derived progenitor cell would be accomplished by gene or protein delivery using methods known to those skilled in the art such as plasmid transfection, viral infection, or membrane-permeant fusion proteins.

B. Biomarkers for an Adult Cardiac-Derived Progenitor Cell

A biomarker useful in the present invention is any detectable molecule or functional assay that can be used to characterize, identify, distinguish, isolate, select or separate an adult cardiac-derived progenitor cell of the present invention from other cells. The biomarker to be measured in the method of the invention includes a gene, a protein, or a variant or a fragment thereof, that can be used to characterize, identify, distinguish, isolate, select or separate an adult cardiac-derived progenitor cell to form a substantially pure population of said cells. Such biomarkers include DNA comprising the entire or partial sequence of the nucleic acid sequence encoding a biomarker, or the complement of such a sequence. Biomarker nucleic acids useful in the invention should be considered to include both DNA and RNA, including mRNA, comprising the entire or partial sequence of any of the nucleic acid sequences of interest. A biomarker protein should be considered to comprise the entire or partial amino acid sequence of any of the biomarker proteins or polypeptides.

The present invention should also be construed to encompass naturally occurring “mutants,” “derivatives,” and “variants” of the biomarker proteins disclosed herein (or of the DNA encoding the same) which mutants, derivatives and variants are altered in one or more amino acids (or, when referring to the nucleotide sequence encoding the same, are altered in one or more base pairs) such that the resulting peptide (or DNA) is not identical to any sequences, but has the same biological property as the biomarker proteins disclosed herein, in that the proteins have indistinguishable biological/biochemical properties. A biological property of the polypeptides of the present invention is the ability of the biomarkers to characterize, identify, distinguish, isolate, select or separate an adult cardiac-derived progenitor cell.

A nuclear biomarker may be used to practice certain aspects of the invention. By “nuclear biomarker” is intended a biomarker that is predominantly expressed in the nucleus of the cell. A nuclear biomarker may be expressed to a lesser degree in other parts of the cell.

The biomarkers to be measured in the methods of the invention include any gene, nucleic acid, protein, or metabolite that can be used to characterize, identify, distinguish, isolate, select or separate an adult cardiac-derived progenitor cell, as defined herein. Biomarkers of particular interest include genes and proteins involved in cardiac development, mesenchymal stem cell markers, and transporter molecules. In one embodiment, the biomarker is an enzyme, a receptor/ligand, a transporter, a protein associated with cellular differentiation, or a transcription factor.

A cell of the present invention may be characterized as “positive” for a particular biomarker. A cell positive for a biomarker is one wherein a cell of the invention expresses a specific biomarker protein, or a nucleic acid encoding said protein,. In one embodiment, a cell of the invention is selected because it expresses an Abcg2 protein, or a nucleic acid encoding Abcg2.

A cell of the present invention may be characterized as “negative” for a particular biomarker. A cell negative for a biomarker is one wherein a cell of the invention does not express a detectable specific biomarker protein, or a nucleic acid encoding said protein. In one embodiment, a cell of the present invention does not express c-kit protein, or a nucleic acid encoding c-kit.

A cell of the present invention may be selected based upon a combination of positive and negative biomarkers. In one embodiment, a biomarker for an adult cardiac-derived progenitor cell is telomerase reverse transcriptase (TERT), an RNA-dependent DNA polymerase that maintains the lariat like loop (telomere) that caps chromosome ends.

In another embodiment, a biomarker for an adult cardiac-derived progenitor cell is a cell's ability to efflux Hoechst Dyes, including but not limited to, Hoechst 33342 or Hoechst 33258.

In still another embodiment, a biomarker for an adult cardiac-derived progenitor cell is an ATP-binding cassette (ABC) transporter protein or a nucleic acid encoding an ATP-binding cassette (ABC) transporter that mediates dye efflux, including Abcg2 (also known as breast cancer resistance protein, or BCRP).

In yet anther embodiment, a biomarker for an adult cardiac-derived progenitor cell is an ATP-binding cassette (ABC) transporter protein or a nucleic acid encoding an ATP-binding cassette (ABC) transporter that mediates dye efflux, including MDR1 (multi-drug resistance-1/Pglycoprotein/Abcb1).

In another embodiment, a biomarker for an adult cardiac-derived progenitor cell is a cardiogenic transcription factor protein or a nucleic acid encoding at least one cardiogenic transcription factor selected from the group including, but not limited to, Mef2a, dHand, Gata4, Tbx2, Tbx5, and Tbx20.

In yet another embodiment, a biomarker for an adult cardiac-derived progenitor cell is at least one mesenchymal stem cell marker protein or a nucleic acid encoding a mesenchymal stem cell marker selected from the group including CD105, CD90, and CD44.

In still another embodiment, a cell of the invention does not express a biomarker comprising c-kit.

In still another embodiment, a cell of the invention does not express any one biomarker comprising a hematopoietic stem cell marker such as CD45, CD34, and CD31 or a hematopoietic transcription factor such as Lin, Lmo2, Gata2, and Tal.

It will be appreciated by a skilled artisan, that a biomarker of the present invention may be a receptor, a ligand, a transporter, a protein associated with cellular differentitation, or any cell surface protein that identifies an adult cardiac-derived progenitor cell. A biomarker of the present invention is not limited to those disclosed herein, but includes any marker, both known and unknown, that identifies an adult cardiac-derived progenitor cell of the present invention. In still another embodiment, a biomarker for an adult cardiac-derived progenitor cell is an extracellular protein or bioinformatically predicted extracellular protein present in an adult cardiac-derived progenitor cell that had been identified using other criteria.

Although the methods of the invention require the detection of at least one positive biomarker expressed by a cell, two, three, four, five, six, seven, eight, nine, ten, or more biomarkers may be used to characterize the cell. It is recognized that detection of more than one biomarker on the surface of a cell may allow for the selection of a more refined cell population. Therefore, in some embodiments, two or more biomarkers are used; more preferably, two or more complementary biomarkers are used to characterize and isolate the cell of the invention.

C. Biomarkers as Target Molecules

It is understood by those of skill in the art that a biomarker used to characterize, identify, distinguish, isolate, select or separate an adult cardiac-derived progenitor cell generally refers to a protein or a nucleic acid encoding a protein that can be detected using any number of methods presently available to the skilled artisan, or that become available in the future. Accordingly, a biomarker of the present invention functions as a target molecule in the methods of the invention. A target molecule, as used herein, is a nucleic acid or protein that interacts with a targeting moiety to identify, distinguish, isolate, select, or separate an adult cardiac-derived progenitor cell from other cells according to the method of the invention. The invention should in no way be considered to be limited to any one or more biomarker detection method.

1. Targeting Moiety

A target molecule is characterized by its interaction with a targeting moiety. A “targeting moiety,” as used herein, may be an antibody, a naturally-occurring ligand for a receptor or functional derivatives thereof, a vitamin, a small molecule mimetic of a naturally-occurring ligand, a peptidomimetic, a polypeptide or aptamer, or any other molecule provided it binds specifically to a target molecule, or a fragment thereof.

A targeting moiety may, either directly or indirectly, comprise a detectable tag or label, thereby labeling a cell comprising a target molecule of interest. Labeling may be accomplished by any method known to those of skill in the art. For example, an antibody directed to a target molecule may comprise a detectable tag or label. In another example, an antibody directed to a target molecule may be contacted by a second antibody comprising a detectable tag or label.

The labels used are those which are suitable for use in systems in which cells are to be analyzed or sorted based upon the attachment of a label to a target molecule via a targeting moiety. The label may be fluorochromated anti-target molecule antibody, which may include but is not limited to a magnetic bead-, colloidal bead-, FITC-, AMCA-, fluorescent particle-, or liposome-conjugated antibodies.

2. Targeting Moiety—Antibodies

When the antibody used as a targeting moiety in the compositions and methods of the invention is a polyclonal antibody (IgG), the antibody is generated by inoculating a suitable animal with the targeted cell surface molecule. Antibodies produced in the inoculated animal which specifically bind to the cell surface molecule are then isolated from fluid obtained from the animal. Antibodies may be generated in this manner in several non-human mammals such as, but not limited to goat, sheep, horse, camel, rabbit, and donkey. Methods for generating polyclonal antibodies are well known in the art and are described, for example in Harlow, et al. (1999, In: Antibodies, A Laboratory Manual, Cold Spring Harbor, N.Y.).

Monoclonal antibodies directed against a full length targeted cell surface molecule or fragments thereof may be prepared using any well known monoclonal antibody preparation procedures, such as those described, for example, in Harlow et al. (1999, In: Antibodies, A Laboratory Manual, Cold Spring Harbor, N.Y.) and in Tuszynski et al. (1988, Blood, 72:109-115). Human monoclonal antibodies may be prepared by the method described in U.S. Patent Publication 2003/0224490. Monoclonal antibodies directed against an antigen are generated from mice immunized with the antigen using standard procedures as referenced herein. Nucleic acid encoding the monoclonal antibody obtained using the procedures described herein may be cloned and sequenced using technology which is available in the art, and is described, for example, in Wright et al. (1992, Critical Rev. in Immunol. 12(3,4):125-168) and the references cited therein.

When the antibody used in the methods of the invention is a biologically active antibody fragment or a synthetic antibody corresponding to antibody to a targeted cell surface molecule, the antibody is prepared as follows: a nucleic acid encoding the desired antibody or fragment thereof is cloned into a suitable vector. The vector is transfected into cells suitable for the generation of large quantities of the antibody or fragment thereof. DNA encoding the desired antibody is then expressed in the cell thereby producing the antibody. The nucleic acid encoding the desired peptide may be cloned and sequenced using technology which is available in the art, and described, for example, in Wright et al. (1992, Critical Rev. in Immunol. 12(3,4):125-168) and the references cited therein. Alternatively, quantities of the desired antibody or fragment thereof may also be synthesized using chemical synthesis technology. If the amino acid sequence of the antibody is known, the desired antibody can be chemically synthesized using methods known in the art as described elsewhere herein.

The present invention also includes the use of humanized antibodies specifically reactive with targeted cell surface molecule epitopes. These antibodies are capable of binding to the targeted cell surface molecule. The humanized antibodies useful in the invention have a human framework and have one or more complementarity determining regions (CDRs) from an antibody, typically a mouse antibody, specifically reactive with a targeted cell surface molecule.

When the antibody used in the invention is humanized, the antibody can be generated as described in Queen, et al. (U.S. Pat. No. 6,180,370), Wright et al., (supra) and in the references cited therein, or in Gu et al. (1997, Thrombosis and Hematocyst 77(4):755-759), or using other methods of generating a humanized antibody known in the art. The method disclosed in Queen et al. is directed in part toward designing humanized immunoglobulins that are produced by expressing recombinant DNA segments encoding the heavy and light chain complementarity determining regions (CDRs) from a donor immunoglobulin capable of binding to a desired antigen, attached to DNA segments encoding acceptor human framework regions. Generally speaking, the invention in the Queen patent has applicability toward the design of substantially any humanized immunoglobulin. Queen explains that the DNA segments will typically include an expression control DNA sequence operably linked to the humanized immunoglobulin coding sequences, including naturally-associated or heterologous promoter regions. The expression control sequences can be eukaryotic promoter systems in vectors capable of transforming or transfecting eukaryotic host cells or the expression control sequences can be prokaryotic promoter systems in vectors capable of transforming or transfecting prokaryotic host cells. Once the vector has been incorporated into the appropriate host, the host is maintained under conditions suitable for high level expression of the introduced nucleotide sequences and as desired the collection and purification of the humanized light chains, heavy chains, light/heavy chain dimers or intact antibodies, binding fragments or other immunoglobulin forms may follow (Beychok, Cells of Immunoglobulin Synthesis, Academic Press, New York, (1979), which is incorporated herein by reference).

Human constant region (CDR) DNA sequences from a variety of human cells can be isolated in accordance with well known procedures. Preferably, the human constant region DNA sequences are isolated from immortalized B-cells as described in WO 87/02671. CDRs useful in producing the antibodies of the present invention may be similarly derived from DNA encoding monoclonal antibodies capable of binding to the targeted cell surface molecule. Such humanized antibodies may be generated using well known methods in any convenient mammalian source capable of producing antibodies, including, but not limited to, mice, rats, camels, llamas, rabbits, or other vertebrates. Suitable cells for constant region and framework DNA sequences and host cells in which the antibodies are expressed and secreted, can be obtained from a number of sources, such as the American Type Culture Collection, Manassas, Va.

One of skill in the art will further appreciate that the present invention encompasses the use of antibodies derived from camelid species. That is, the present invention includes, but is not limited to, the use of antibodies derived from species of the camelid family. As is well known in the art, camelid antibodies differ from those of most other mammals in that they lack a light chain, and thus comprise only heavy chains with complete and diverse antigen binding capabilities (Hamers-Casterman et al., 1993, Nature, 363:446-448). Such heavy-chain antibodies are useful in that they are smaller than conventional mammalian antibodies, they are more soluble than conventional antibodies, and further demonstrate an increased stability compared to some other antibodies. Camelid species include, but are not limited to Old World camelids, such as two-humped camels (C. bactrianus) and one humped camels (C. dromedarius). The camelid family further comprises New World camelids including, but not limited to llamas, alpacas, vicuna and guanaco. The production of polyclonal sera from camelid species is substantively similar to the production of polyclonal sera from other animals such as sheep, donkeys, goats, horses, mice, chickens, rats, and the like. The skilled artisan, when equipped with the present disclosure and the methods detailed herein, can prepare high-titers of antibodies from a camelid species. As an example, the production of antibodies in mammals is detailed in such references as Harlow et al., (1999, Using Antibodies: A Laboratory Manual, Cold Spring Harbor Laboratory Press, NY).

V_{H} proteins isolated from other sources, such as animals with heavy chain disease (Seligmann et al., 1979, Immunological Rev. 48:145-167, incorporated herein by reference in its entirety), are also useful in the compositions and methods of the invention. The present invention further comprises variable heavy chain immunoglobulins produced from mice and other mammals, as detailed in Ward et al. (1989, Nature 341:544-546, incorporated herein by reference in its entirety). Briefly, V_{H} genes are isolated from mouse splenic preparations and expressed in E. coli. The present invention encompasses the use of such heavy chain immunoglobulins in the compositions and methods detailed herein.

Antibodies useful as targeting moieties in the invention may also be obtained from phage antibody libraries. To generate a phage antibody library, a cDNA library is first obtained from mRNA which is isolated from cells, e.g., the hybridoma, which express the desired protein to be expressed on the phage surface, e.g., the desired antibody. cDNA copies of the mRNA are produced using reverse transcriptase. cDNA which specifies immunoglobulin fragments are obtained by PCR and the resulting DNA is cloned into a suitable bacteriophage vector to generate a bacteriophage DNA library comprising DNA specifying immunoglobulin genes. The procedures for making a bacteriophage library comprising heterologous DNA are well known in the art and are described, for example, in Sambrook et al. (2001, Molecular Cloning: A Laboratory Manual, Cold Spring Harbor Laboratory Press, Cold Spring Harbor, N.Y.).

One of skill in the art will appreciate that it may be desirable to detect more than one protein of interest in a biological sample. Therefore, in particular embodiments, at least two antibodies directed to two distinct proteins are used. Where more than one antibody is used, these antibodies may be added to a single sample sequentially as individual antibody reagents or simultaneously as an antibody cocktail. Alternatively, each individual antibody may be added to a separate sample from the same source, and the resulting data pooled.

3. Targeting Moieties—Protein, Peptide, and Polypeptide

Other types of targeting moieties useful in the invention comprise a protein, peptide or polypeptide and may be obtained using standard methods known to the skilled artisan. Such methods include chemical organic synthesis or biological means. Biological means include purification from a biological source, recombinant synthesis and in vitro translation systems, using methods well known in the art.

A peptide may be chemically synthesized by Merrifield-type solid phase peptide synthesis. This method may be routinely performed to yield peptides up to about 60-70 residues in length, and may, in some cases, be utilized to make peptides up to about 100 amino acids long. Larger peptides may also be generated synthetically via fragment condensation or native chemical ligation (Dawson et al., 2000, Ann. Rev. Biochem. 69:923-960). An advantage to the utilization of a synthetic peptide route is the ability to produce large amounts of peptides, even those that rarely occur naturally, with relatively high purities, i.e., purities sufficient for research, diagnostic or therapeutic purposes.

Solid phase peptide synthesis is described by Stewart et al. in Solid Phase Peptide Synthesis, 2nd Edition, 1984, Pierce Chemical Company, Rockford, Illinois; and Bodanszky and Bodanszky in The Practice of Peptide Synthesis, 1984, Springer-Verlag, New York. At the outset, a suitably protected amino acid residue is attached through its carboxyl group to a derivatized, insoluble polymeric support, such as cross-linked polystyrene or polyamide resin. “Suitably protected” refers to the presence of protecting groups on both the α-amino group of the amino acid, and on any side chain functional groups. Side chain protecting groups are generally stable to the solvents, reagents and reaction conditions used throughout the synthesis, and are removable under conditions which will not affect the final peptide product. Stepwise synthesis of the oligopeptide is carried out by the removal of the N-protecting group from the initial amino acid, and coupling thereto of the carboxyl end of the next amino acid in the sequence of the desired peptide. This amino acid is also suitably protected. The carboxyl of the incoming amino acid can be activated to react with the N-terminus of the support-bound amino acid by formation into a reactive group, such as formation into a carbodiimide, a symmetric acid anhydride, or an “active ester” group, such as hydroxybenzotriazole or pentafluorophenyl esters.

Examples of solid phase peptide synthesis methods include the BOC method, which utilizes tert-butyloxcarbonyl as the α-amino protecting group, and the FMOC method, which utilizes 9-fluorenylmethyloxcarbonyl to protect the α-amino of the amino acid residues. Both methods are well-known by those of skill in the art.

Incorporation of N- and/or C-blocking groups may also be achieved using protocols conventional to solid phase peptide synthesis methods. For incorporation of C-terminal blocking groups, for example, synthesis of the desired peptide is typically performed using, as solid phase, a supporting resin that has been chemically modified so that cleavage from the resin results in a peptide having the desired C-terminal blocking group. To provide peptides in which the C-terminus bears a primary amino blocking group, for instance, synthesis is performed using a p-methylbenzhydrylamine (MBHA) resin, so that, when peptide synthesis is completed, treatment with hydrofluoric acid releases the desired C-terminally amidated peptide. Similarly, incorporation of an N-methylamine blocking group at the C-terminus is achieved using N-methylaminoethyl-derivatized DVB (divinylbenzene), resin, which upon hydrofluoric acid (HF) treatment releases a peptide bearing an N-methylamidated C-terminus. Blockage of the C-terminus by esterification can also be achieved using conventional procedures. This entails use of resin/blocking group combination that permits release of side-chain peptide from the resin, to allow for subsequent reaction with the desired alcohol, to form the ester function. FMOC protecting group, in combination with DVB resin derivatized with methoxyalkoxybenzyl alcohol or equivalent linker, can be used for this purpose, with cleavage from the support being effected by trifluoroacetic acid (TFA) in dicholoromethane. Esterification of the suitably activated carboxyl function, e.g. with dicyclohexylcarbodiimide (DCC), can then proceed by addition of the desired alcohol, followed by de-protection and isolation of the esterified peptide product.

Incorporation of N-terminal blocking groups may be achieved while the synthesized peptide is still attached to the resin, for instance by treatment with a suitable anhydride and nitrile. To incorporate an acetyl blocking group at the N-terminus, for instance, the resin-coupled peptide can be treated with 20% acetic anhydride in acetonitrile. The N-blocked peptide product may then be cleaved from the resin, de-protected and subsequently isolated.

Prior to its use as a targeting moiety, a peptide may be purified to remove contaminants. In this regard, it will be appreciated that the peptide can be purified so as to meet the standards set out by the appropriate regulatory agencies. Any one of a number of a conventional purification procedures may be used to attain the required level of purity including, for example, reversed-phase high-pressure liquid chromatography (HPLC) using an alkylated silica column such as C₄-, C₈- or C₁₈-silica. A gradient mobile phase of increasing organic content is generally used to achieve purification, for example, acetonitrile in an aqueous buffer, usually containing a small amount of trifluoroacetic acid. Ion-exchange chromatography can be also used to separate polypeptides based on their charge. Affinity chromatography is also useful in purification procedures.

Antibodies and other peptide targeting moieties may be modified using ordinary molecular biological techniques to improve their resistance to proteolytic degradation or to optimize solubility properties or to render them more suitable as a therapeutic agent. Analogs of such polypeptides include those containing residues other than naturally occurring L-amino acids, e.g., D-amino acids or non-naturally occurring synthetic amino acids. The polypeptides useful in the invention may further be conjugated to non-amino acid moieties that are useful in their application. In particular, moieties that improve the stability, biological half-life, water solubility, and immunologic characteristics of the peptide are useful. A non-limiting example of such a moiety is polyethylene glycol (PEG).

4. Targeting Moieties—Nucleic Acids

Any number of procedures may be used for the generation of targeting moieties comprising nucleic acids using recombinant DNA methodology well known in the art (see Sambrook et al., 2001, Molecular Cloning: A Laboratory Manual, Cold Spring Harbor Laboratory Press, New York; Ausubel et al., 2001, Current Protocols in Molecular Biology, Green & Wiley, New York) and by direct synthesis. For recombinant and in vitro transcription, DNA encoding RNA molecules can be obtained from known clones, by synthesizing a DNA molecule encoding an RNA molecule, or by cloning the gene encoding the RNA molecule. Techniques for in vitro transcription of RNA molecules and methods for cloning genes encoding known RNA molecules are described by, for example, Sambrook et al. (2001, Molecular Cloning: A Laboratory Manual, Cold Spring Harbor Laboratory Press, Cold Spring Harbor, N.Y.).

An isolated nucleic acid targeting moiety of the present invention can be produced using conventional nucleic acid synthesis or by recombinant nucleic acid methods known in the art and described elsewhere herein (Sambrook et al., 2001, Molecular Cloning: A Laboratory Manual, Cold Spring Harbor Laboratory Press, Cold Spring Harbor, N.Y.) and Ausubel et al. (2001, Current Protocols in Molecular Biology, Green & Wiley, New York).

As an example, a method for synthesizing nucleic acids de novo involves the organic synthesis of a nucleic acid from nucleoside derivatives. This synthesis may be performed in solution or on a solid support. One type of organic synthesis is the phosphotriester method, which has been used to prepare gene fragments or short genes. In the phosphotriester method, oligonucleotides are prepared which can then be joined together to form longer nucleic acids. For a description of this method, see Narang, et al., (1979, Meth. Enzymol., 68: 90) and U.S. Pat. No. 4,356,270. The phosphotriester method can be used in the present invention to synthesize an isolated snRNA.

In addition, a nucleic acid targeting moiety of the present invention can be synthesized in whole or in part, or an isolated snRNA can be conjugated to another nucleic acid using organic synthesis such as the phosphodiester method, which has been used to prepare a tRNA gene. See Brown, et al. (1979, Meth. Enzymol., 68: 109) for a description of this method. As in the phosphotriester method, the phosphodiester method involves synthesis of oligonucleotides which are subsequently joined together to form the desired nucleic acid.

A third method for synthesizing a nucleic acid targeting moiety (U.S. Pat. No. 4,293,652) is a hybrid of the above-described organic synthesis and molecular cloning methods. In this process, the appropriate number of oligonucleotides to make up the desired nucleic acid sequence is organically synthesized and inserted sequentially into a vector which is amplified by growth prior to each succeeding insertion.

In addition, molecular biological methods, such as using a nucleic acid as a template for a PCR or LCR reaction, or cloning a nucleic acid into a vector and transforming a cell with the vector can be used to make large amounts of the nucleic acid of the present invention.

C. Pharmaceutical Compositions

The present invention includes pharmaceutical compositions comprising a cell of the invention. In one embodiment, the present invention includes a pharmaceutical composition comprising a substantially pure population of cells of the invention selected according to the methods of the present invention. Compositions comprising an adult cardiac-derived progenitor cell can be incorporated into pharmaceutical compositions suitable for administration to a subject, preferably a human. The cells are preferably human cells.

As used herein, the language “pharmaceutically acceptable carrier” is intended to include any and all solvents, dispersion media, coatings, antibacterial and antifungal agents, isotonic and absorption delaying agents, and the like, compatible with pharmaceutical administration. Supplementary active compounds can also be incorporated into the compositions.

A formulated composition comprising a cardiac-derived progenitor cell can assume a variety of states. Generally, the composition comprising a cardiac-derived progenitor cell is formulated in a manner that is compatible with the intended method of administration.

A composition comprising a cardiac-derived progenitor cell can be formulated in combination with another agent, e.g., another therapeutic agent or an agent that stabilizes the composition comprising a cardiac-derived progenitor cell e.g., a protein, chelators, e.g., EDTA (e.g., to remove divalent cations such as Mg²⁺), salts, RNAse inhibitors (e.g., a broad specificity RNAse inhibitor such as RNAsin) and so forth.

In another embodiment, the composition comprising a cardiac-derived progenitor cell is administered in conjunction with another therapeutic agent such as an antibiotic, an antiviral, an anti-inflammatory, a chemotherapeutic agent, an immunosuppressive agent, a beta blocker drug, a calcium channel blocker, a diuretic, a drug that increases blood flow to the heart such as nitroglycerine or a nitrate, an angiotensin converting enzyme (ACE) inhibitor, a drug which lowers cholesterol, and a drug that lowers blood pressure, or any other therapeutic compound useful in the treatment of a particular disease or disorder, especially therapeutic agents useful in treating cardiac disease. The composition comprising a cardiac-derived progenitor cell may be administered as part of an on-going treatment regimen or therapy for a particular disease such as myocardial infarction, atherosclerosis, angina, coronary artery disease, cardiac hypertrophy, or heart failure.

Pharmaceutical compositions of the invention include a pharmaceutical carrier that may contain a variety of components that provide a variety of functions, including regulation of drug concentration, regulation of solubility, chemical stabilization, regulation of viscosity, absorption enhancement, regulation of pH, and the like. The pharmaceutical carrier may comprise a suitable liquid vehicle or excipient and an optional auxiliary additive or additives. The liquid vehicles and excipients are conventional and commercially available. Illustrative thereof are distilled water, physiological saline, aqueous solutions of dextrose, and the like. For water soluble formulations, the pharmaceutical composition preferably includes a buffer such as a phosphate buffer, or other organic acid salt, preferably at a pH of between about 7 and 8. Other components may include antioxidants, such as ascorbic acid, hydrophilic polymers, such as, monosaccharides, disaccharides, and other carbohydrates including cellulose or its derivatives, dextrins, chelating agents, such as EDTA, and like components well known to those in the pharmaceutical sciences, e.g., Remington's Pharmaceutical Science, latest edition (Mack Publishing Company, Easton, Pa.).

An adult cardiac-derived progenitor cell of the invention may be administered into a recipient in a wide variety of ways. Preferred modes of administration are parenteral, intravenous, intra-arterial, intracardiac, intramuscular, surgical implant, infusion pump, or via catheter.

### II. Methods

A. Obtaining a Cell of the Invention

A cell of the invention is obtained from a live sample procured from a human heart. In the case of an autologous cell donation, a cell of the invention is obtained from a fresh surgical sample, such as a cardiac biopsy performed for clinical indications. In the case of an allogenic cell donation, a cell of the invention may be obtained from a surgical sample, such as a cardiac biopsy from a patient undergoing therapeutic transplantation or a donor heart not utilized for transplantation. The surgical sample or biopsy may be obtained from the right ventricle (RV), interventricular septum, left ventricle (LV), or any other region of the cardiac tissue that comprises cardiac progenitor cells. In one embodiment, the surgical sample is about 1 to about 5 grams in size. In another embodiment, the surgical sample is less than 1 gram in size.

The cells described herein can be obtained by mechanically and enzymatically dissociating cells from human myocardial tissue present in the sample. Mechanical dissociation can be brought about using methods that include, without limitation, chopping and/or mincing the tissue, and/or centrifugation and the like. Enzymatic dissociation of connective tissue and from cell-to-cell associations can be brought about by enzymes including, but not limited to, Blendzyme, DNAse I, collegenase and trypsin, or a cocktail of enzymes found to be effective in liberating cells from the cardiac sample. The procedure for mechanically and enzymatically isolating a cell of the present invention should not be construed to be limited to the materials and techniques presented herein, but rather it will be recognized that these techniques are well-established and fall well within the scope of experimental optimization performed routinely in the art.

B. Methods of Selecting and Characterizing an Adult Cardiac-Derived Progenitor

Cell

It will be appreciated by one skilled in the art that it is often desirable to select one cell type from a mixture of cells.

“Cell selection,” as used herein, refers to the method of obtaining a cell of the invention according to biomarkers (target molecules) present or absent on that cell.

The method of cell selection, described herein, arrives at an adult cardiac-derived progenitor cell of the invention, namely wherein the selected cell expresses at least one of the following: a detectable amount of Abcg2 protein or an mRNA encoding Abcg2 (Abcg2^{pos}); a detectable amount of MDR1 protein or an mRNA encoding MDR1 (MDR1^{pos}); a detectable amount of Mef2a protein or an mRNA encoding Mef2A (Mef2a^{pos}); a detectable amount of Gata4 protein or an mRNA encoding Gata4 (Gata4^{pos}); a detectable amount of dHAND protein or an mRNA encoding dHAND (dHAND^{pos}); a detectable amount of Tbx2 protein or an mRNA encoding Tbx2 (Tbx2^{pos}); a detectable amount of Tbx5 protein or an mRNA encoding Tbx5 (Tbx5^{pos}); a detectable amount of Tbx20 protein or an mRNA encoding Tbx20 (Tbx20^{pos}); a detectable amount of CD105 protein or an mRNA encoding CD105 (CD105^{pos}); a detectable amount of CD90 protein or an mRNA encoding CD90 (CD90^{pos}); a detectable amount of CD44 protein or an mRNA encoding CD44 (CD44^{pos}).

The method of cell selection, described herein, arrives at an adult cardiac-derived progenitor cell of the invention, namely wherein the selected cell does not express at least one of the following: a detectable amount of c-kit protein or an mRNA encoding c-kit (c-kit^{neg}); a detectable amount of Lin protein or an mRNA encoding Lin (Leg); a detectable amount of Lmo2 protein or an mRNA encoding Lmo2 (Lmo2^{neg}); a detectable amount of Gata2 protein or an mRNA encoding Gata2 (Gata^{neg}); a detectable amount of Tal protein or an mRNA encoding Tal (Tal^{neg}); a detectable amount of CD45 protein or an mRNA encoding CD45 (CD45^{neg}); a detectable amount of CD31 protein or an mRNA encoding CD31 (CD31^{neg}); a detectable amount of CD34 protein or an mRNA encoding CD34 (CD34^{neg}). It will be appreciated by one skilled in the art that cell selection can occur at any stage after procurement of a sample and prior to administration an adult cardiac-derived progenitor cell to a human. In one embodiment, cell selection is performed once. In another embodiment, cell selection is performed two or more times at various times throughout in vitro processing of a cardiac sample.

The invention includes cell selection using a single target molecule to characterize, identify, distinguish, isolate, select or separate an adult cardiac-derived progenitor cell from other cells. The invention further includes cell selection using one or more target molecules to characterize, identify, distinguish, isolate, select or separate an adult cardiac-derived progenitor cell from other cells.

When multiple target molecules are used in the process of cell selection, each target molecule may be used singly in sequential rounds of cell selection, in combination in a single cell selection step, or in combination in a sequential series of cell selection steps to arrive at a substantially pure cell population enriched for the target molecules used in cell selection.

By way of a non-limiting example, in one embodiment of the present invention, a population of cells may be selected using a single target molecule, i.e. Abcg2. This population of cells may be subjected to subsequent rounds of cell selection that also select for Abcg2, thereby arriving at a substantially pure population of cells enriched for Abcg2^{pos} cells. In another embodiment, a population of cells may be selected for a single target molecule, i.e. Abcg2. This population of cells may be further selected for other target molecules identified herein, such as MDR1, mesenchymal stem cell markers, or the absence of c-kit.

The present invention contemplates iterative steps of selection to arrive at a substantially pure population of adult cardiac-derived progenitor cells. In one embodiment, the selected cells are substantially Abcg2^{pos}. In another embodiment, the selected cells are substantially MDR1^{pos}. In still another embodiment the selected cells are substantially CD105^{pos}, CD90^{pos}, CD44^{pos}, or any combination thereof. In yet another embodiment, the selected cells are substantially Mef2a^{pos}, Gata4^{pos}, dHand^{pos}, Tbx2^{pos}, Tbx5^{pos}, Tbx20^{pos}, or any combination thereof. In still another embodiment of the invention, the selected cells are Abcg2^{pos}, MDR1^{pos}, CD105^{pos}, CD90^{pos}, CD44^{pos}, Mef2a^{pos}, Gata4^{pos}, dHand^{pos}, Tbx2^{pos}, Tbx5^{pos}, Tbx20^{pos}, or any combination thereof.

In another embodiment of the present invention, the selected cells are substantially c-kit^{neg}. In another embodiment, the selected cells are substantially CD31^{neg}, CD34^{neg}, CD45^{neg}, or any combination thereof. In another embodiment, the selected cells are substantially Lin^{neg}, Lmo^{neg}, Gata2^{neg}, Tal^{neg}, or any combination thereof. In another embodiment, the selected cells are substantially c-kit^{neg}, CD31^{neg}, CD34^{neg}, CD45^{neg}, Lin^{neg}, Lmo^{neg}, Gata2^{neg}, Tal^{neg}, or any combination thereof.

In another embodiment, the selected cells are substantially Abcg2^{pos} and c-kit^{neg}. In another embodiment of the invention, the selected cells are substantially MDR1^{pos} and c-kit^{neg}g. In another embodiment, the selected cells are substantially Abcg2^{pos}, MDR1^{pos}, CD105^{pos}, CD90^{pos}, CD44^{pos}, Mef2a^{pos}, Gata4^{pos}, dHand^{pos}, Tbx2^{pos}, Tbx5^{pos}, Tbs20^{pos}, c-kit^{neg}, CD31^{neg}, CD34^{neg}, CD45^{neg}, Lin^{neg}, Lmo^{neg}, Gata2^{neg}, Tal^{neg}, or any combination thereof.

1. Cell Sorting: Fluorescence Activated Cell Sorting (FACS)

In one embodiment of the invention, the adult cardiac-derived cells of the invention are sorted according to the expression of at least one biomarker using techniques available to sort cells. Analysis of the cell population and cell sorting based upon the presence of the label can be accomplished using a number of techniques known in the art. Cells can be analyzed or sorted by, for example, flow cytometry. These techniques allow the analysis and sorting according to one or more parameters of the cells. Usually one or multiple parameters can be analyzed simultaneously in combination with other measurable parameters of the cell, including, but not limited to, cell type, cell surface markers, DNA content, etc. The data can be analyzed and cells are sorted using any formula or combination of the measured parameters. Cell sorting and cell analysis methods are known in the art and are described in, for example, The Handbook of Experimental Immunology, Volumes 1 to 4, (D. N. Weir, editor); Flow Cytometry Cell Sorting (A. Radbruch, editor, Springer Verlag, 1992); and Cell Separation Methods and Applications (D. Recktenwald and A. Radbruch, eds., 1997) Marcel Dekker, Inc. N.Y. Cells can also be analyzed using microscopy techniques including, for example, laser scanning microscopy, spinning disc microscopy, fluorescence microscopy; techniques such as these can also be used in combination with image analysis systems. Other methods for cell sorting include, for example, panning and separation using affinity techniques, including those techniques using solid supports such as plates, beads and columns.

FACS permits the separation of subpopulations of cells initially on the basis of their light scatter properties as they pass through a laser beam. Since cells are tagged with fluorescent-labeled product, they are characterized by fluorescence intensity and positive and negative windows set on the FACS to collect label⁺ (bright fluorescence) and label⁻ (low fluorescence) cells. Positive and negative windows are set to collect label⁺ and label⁻ cells, respectively. Positve cells, negative cells, and subpopulations co-expressing various combinations of markers are collected.

Identification of antibody-expressing bacteria by FACS is directly based on the affinity for the soluble hapten thus eliminating artifacts due to binding on solid surfaces. This means only the high affinity antibodies are recovered by sorting following binding of low concentrations of fluorescently labeled antigen. There is no analogous method for specifically selecting phage with very high affinity. Additionally, the sorting of positive clones is essentially quantitative. It is limited only by the accuracy of the flow cytometer, which is on the order of 95%. In contrast with phage technology, the efficiency of selection is not limited by avidity effects because screening does not depend on binding to a surface having multiple antigens and thus the potential for multivalent attachment sites.

2. Cell Sorting: Magnetic Cell Sorting (MACS)

Some methods for cell sorting utilize magnetic separations, and some of these methods utilize magnetic beads. Different magnetic beads are available from a number of sources, including for example, Dynal (Norway), Advanced Magnetics (Cambridge, Mass., U.S.A.), Immuncon (Philadelphia, U.S.A.), Immunotec (Marseilles, France), and Miltenyi Biotec GmbH (Germany).

Preferred magnetic labeling methods include colloidal superparamagnetic particles in a size range of 5 to 200 rim, preferably in a size of 10 to 100 nm. These magnetic particles allow a quantitative magnetic labeling of cells, thus the amount of coupled magnetic label is proportional to the amount of bound product, and the magnetic separation methods are sensitive to different amounts of product secretion. Colloidal particles with various specificities are known in the art, and are available, for example, through Miltenyi Biotec GmbH. The use of immuno specific fluorescent or magnetic liposomes can also be used for quantitative labeling of captured product. In these cases, the liposomes contain magnetic material and/or fluorescent dyes conjugated with antibody on their surfaces, and magnetic separation is used to allow optimal separation between nonproducing, low producing, and high producing cells.

The magnetic separation can be accomplished with high efficiency by combining a second force to the attractive magnetic force, causing a separation based upon the different strengths of the two opposed forces. Typical opposed forces are, for example, forces induced by magnetic fluids mixed in the separation medium in the magnetic separation chamber, gravity, and viscous forces induced by flow speed of medium relative to the cell.

Any magnetic separation method, preferably magnetic separation methods allowing quantitative separation will be used. It is also contemplated that different separation methods can be combined, for example, magnetic cell sorting can be combined with FACS, to increase the separation quality or to allow sorting by multiple parameters. Preferred techniques include high gradient magnetic separation (HGMS), a procedure for selectively retaining magnetic materials in a chamber or column disposed in a magnetic field. In one application of this technique the product is labeled by attaching it to a magnetic particle. The attachment is generally through association of the product with a label moiety which is conjugated to a coating on the magnetic particle which provides a functional group for the conjugation. The captured product thus coupled to a magnetic “label”, is suspended in a fluid which is then applied to the chamber. In the presence of a magnetic gradient supplied across the chamber, the magnetically labeled target cell is retained in the chamber; if the chamber contains a matrix, it becomes associated with the matrix. Cells which do not have or have only a low amount of magnetic labels pass through the chamber.

The retained cells can then be eluted by changing the strength of, or by eliminating, the magnetic field or by introducing a magnetic fluid. The selectivity for a captured product is supplied by the label moiety conjugated either directly or indirectly to the magnetic particle or by using a primary antibody and a magnetic particle recognizing the primary antibody. The chamber across which the magnetic field is applied is often provided with a matrix of a material of suitable magnetic susceptibility to induce a high magnetic field gradient locally in the chamber in volumes close to the surface of the matrix. This permits the retention of fairly weakly magnetized particles. Publications describing a variety of HGMS systems are known in the art, and include, for example, U.S. Pat. Nos. 4,452,773, 4,230,685, PCT application WO85/04330, U.S. Pat. No. 4,770,183, and PCT/EP89/01602; systems are also described in U.S. Pat. Nos. 5,411,863; 5,543,289; 5,385,707; and 5,693,539, which are commonly owned, and are hereby incorporated herein by reference in their entirety.

In addition, in other embodiments the processes include labeling the cells that contain the target molecule captured by the targeting moiety, if any. Other embodiments can also include analyzing the cell population to detect labeled cells, if any, and if desired, sorting the labeled cells, if any.

3. Cell Sorting: Separation of Cells by Adsorption onto Supports with Immobilized Target Molecules

In another embodiment of the present invention, cells displaying polypeptides that bind to the desired target molecule may be isolated via selective adsorption onto solid matrices. In this case the cell population displaying the polypeptide library is contacted with a solid support in which the antigen is covalently immobilized via standard chemical immobilization methodologies. Cells that display polypeptides capable of interacting with the immobilized target molecules are retained on the solid support and can be separated from non-binding cells. Following several washes with buffer to remove non-specifically adsorbed cells, the cells that are bound via specific interactions are employed for further studies. Such specifically-bound cells can be dissociated from the solid support either by adding large concentrations of the soluble desired molecule to serve as a competitor or, alternatively by adding growth media to allow the cells to grow. In the latter case the progeny of the bound cells is released from the solid support.

C. Cell Propagation and Expansion

A population of adult cardiac-derived progenitor cells can be expanded in vitro. Adult cardiac-derived progenitor cells can be cultured in any way that enhances their expansion.

As used herein, “expansion” refers to increasing the number of cells under conditions in which the cells do not undergo a significant amount of differentiation.

Expansion may occur when cells are plated onto a layer of feeder cells such as, but not limited to, a monolayer of mesenchymal cells. A mesenchymal feeder monolayer can be commercially prepared, or can be autologously formed. The adult cardiac-derived progenitor cells are loosely attached to the monolayer and can be collected in the medium (e.g., in the supernatant). In addition, a cell of the invention may be expanded by culturing in suspension.

Human adult cardiac-derived progenitor cells may be expanded and propagated on coated dishes using standard tissue culture techniques well-known in the art. Basal media useful in mammalian cell culture are known in the art. Non-limiting examples of basal media useful in the defined culture medium of the invention include Minimum Essential Medium Eagle, ADC-1, LPM (Bovine Serum Albumin-free), F10 (HAM), F12 (HAM), Dulbecco's Modified Eagle Medium (DMEM-without serum), DMEM/F12, DCCM1, DCCM2, RPMI 1640, BGJ Medium (with and without Fitton-Jackson Modification), Basal Medium Eagle (BME-with the addition of Earle's salt base), Yamane, IMEM-20, Glasgow Modification Eagle Medium (GMEM), Leibovitz L-15 Medium, McCoy's 5A Medium, Medium M199 (M199E-with Earle's sale base), Medium M199 (M199H-with Hank's salt base), Minimum Essential Medium Eagle (MEM-E-with Earle's salt base), Minimum Essential Medium Eagle (MEM-H-with Hank's salt base) and Minimum Essential Medium Eagle (MEM-NAA with non essential amino acids), among numerous others, including medium 199, CMRL 1415, CMRL 1969, CMRL 1066, NCTC 135, MB 75261, MAB 8713, DM 145, Williams' G, Neuman & Tytell, Higuchi, MCDB 301, MCDB 202, MCDB 501, MCDB 401, MCDB 411, MDBC 153. A preferred basal medium for use in the present invention is DMEM/F12. These and other useful media are available from GIBCO, Grand Island, N.Y., USA, and Biological Industries, Bet HaEmek, Israel, among others. A number of these media are summarized in Methods in Enzymology, Volume LVIII, “Cell Culture”, pp. 62-72, edited by William B. Jakoby and Ira H. Pastan, published by Academic Press, Inc.

In some embodiments, the culture medium of the invention may further include any components known by the skilled artisan to be useful in the culturing of primate stem cells, excluding feeder cells, conditioned medium and animal serum. In particular, for embryonic stem cell culture, the medium further comprises glutamine, non-essential amino acids and 2-mercaptoethanol. In an embodiment, a serum-free defined medium may include at least one additional growth factor. Growth factors useful in the present invention include, but are not limited to, stem cell factor (SCF), glial cell line-derived neurotrophic factor (GDNF), GDNF-family receptor (including GFRα1), leukemia inhibitory factor (LIF), hepatocyte growth factor (HGF), acidic fibroblast growth factor (aFGF), epidermal growth factor (EGF), insulin-like growth factor (including IGF-1 and IGF-2), keratinocyte growth factor (KGF), nerve growth factor (NGF), transforming growth factor beta (TGF-.beta.), vascular endothelial cell growth factor (VEGF), platelet-derived growth factor (PDGF), transforming growth factor (including TGF-β I through V, as well as the TGF-β superfamily: BMP-1 through 12, GDF-1 through 8, dpp, 60A, BIP, OF), various interleukins (such as IL-1 through IL-18), various colony-stimulating factors (such as granulocyte/macrophage colony-stimulating factor (GM-CSF)), Sonic hedgehog, notch, leptin, hormones, and various interferons (such as IFN-gamma). It is further recognized that additional components may be added to the culture medium, provided they support substantially undifferentiated proliferation of primate stem cells, particularly hESCs, and maintain both pluripotency and karyotype of the cells. Such components may be biologically-relevant lipids, antibiotics, antimycotics, anti-oxidants (reducing agents), amino acids, and other components known to the art for the culture of cells, excluding feeder cells, conditioned medium and animal serum. Biologically-relevant lipids include neutral triglycerides of predominantly unsaturated fatty acids such as linoleic, oleic, palmitic, linolenic, and stearic acid, as well as phospholipids such as phosphatidylethanolamine and phosphatidylcholine. Anti-oxidants useful in the defined medium of the invention include, but are not limited to, β-mercaptoethanol, ascorbic acid, monothioglyceroll and dithiothreitol. Antibiotics that can be added into the medium include, but are not limited to, penicillin and streptomycin. Additionally, components may be added to or removed from the medium to induce, inhibit, or enhance the differentiation process.

Preferably, the components are free of endotoxins. Endotoxins are a pyrogen, which is defined as a substance that can cause a fever response. Endotoxins are also toxic to cells grown in tissue culture conditions.

In preferred embodiments, a medium's endotoxicity, as measured in endotoxin units per milliliter (“eu/ml”), will be less than about 0.1 eu/ml, and, in more preferred embodiments, will be less than about 0.05 eu/ml. In particularly preferred embodiments, the endotoxicity of the base medium will be less than about 0.03 eu/ml. Methods for measuring endotoxicity are known in the art. For example, a preferred method is described in the

“Guideline on Validation of the Limulus Amebocyte Lysate Test as an End-product Endotoxin Test for Human and Animal Parental Drugs, Biological Products and Medical Devices,” published by the U.S. Department of Health and Human Services, FDA, December 1987.

The defined culture medium of the invention may be used as part of a culture system to culture primate stem cells. A culture system of the invention comprises a defined culture medium of the invention and a matrix. Matrices useful in a culture system of the invention include, but are not limited to, fibronectin, collagen, laminin, vibronectin, heparan sulfate, poly-D-lysine, peptides, matrigel and combinations thereof. Examples of fibronectin useful in the invention include, but are not limited to, plasma fibronectin, cellular fibronectin, and synthetic fibronectin. Preferably, collagen is collagen IV. Preferably, the matrix is fibronectin, collagen or a combination thereof. Preferably, the matrix is obtained by recombinant or chemical synthesis, or is obtained from a human biological sample.

Typically, the matrix is applied to the surface of a culturing vessel, such as a culture plate or flask. The culturing vessel further contains the defined culture medium of the invention. Alternatively, the matrix is provided in a soluble form in the defined culture medium.

For use in a culture system of the invention, fibronectin is applied to a surface at between about 5 μg/cm² to about 250 μg/cm². Collagen is applied at between about 20 μg/cm² to about 50 μg/cm². When fibronectin and collagen are used together, the same concentration ranges are suitable. Laminin is applied at between about 20 μg/cm² to about 50 μg/cm². Matrigel is applied at between about 50 μg/cm². Concentrations for use in soluble form can be readily assessed from the art. In addition, the skilled artisan is readily able to optimize matrix concentrations for soluble use without undue experimentation

D. Methods of Use

Embodiments of the present invention include methods of administering to a subject an isolated adult cardiac derived progenitor cell or a pharmaceutical composition comprising an isolated adult cardiac derived progenitor cell to treat cardiovascular disease.

Cardiovascular diseases and/or disorders include, but are not limited to, diseases and/or disorders of the pericardium (i.e., pericardium), heart valves (i.e., incompetent valves, stenosed valves, rheumatic heart disease, mitral valve prolapse, aortic regurgitation), myocardium (coronary artery disease, myocardial infarction, heart failure, ischemic heart disease, angina) blood vessels (i.e., arteriosclerosis, aneurysm) or veins (i.e., varicose veins, hemorrhoids). In specific embodiments, the cardiovascular disease includes, but is not limited to, coronary artery diseases (i.e., arteriosclerosis, atherosclerosis, and other diseases of the arteries, arterioles and capillaries or related complaint), acute myocardial infarct, organizing myocardial infarct, ischemic heart disease, arrhythmia, left ventricular dilatation, emboli, heart failure, congestive heart failure, subendocardial fibrosis, left or right ventricular hypertrophy, and myocarditis. Yet further, one skilled in the art recognizes that cardiovascular diseases and/or disorders can result from congenital defects, genetic defects, environmental influences (i.e., dietary influences, lifestyle, stress, etc.), and other defects or influences.

The methods of the invention comprise providing a substantially pure population of isolated adult cardiac derived progenitor cells of the present invention to a patient diagnosed, suspected of having, or being at risk of a cardiovascular disease or injury. In a preferred embodiment, the isolated adult cardiac derived progenitor cells are isolated from the patient being treated.

In a related embodiment, the present invention includes a method of reconstituting or repopulating dead or injured myocardium in a patient. This method comprises contacting a patient having injured or dead myocardium with a substantially pure population of isolated adult cardiac derived progenitor cells of the present invention. In one embodiment, the patient has previously suffered a myocardial infarction and/or has been diagnosed with congestive heart failure. Cells extracted and purified using methods of the present invention can be used to reconstitute dead myocardium by differentiating into normal components of adult hearts after being transplanted into ischemia-induced heart muscle.

In further embodiments, an isolated adult cardiac derived progenitor cell of the present invention or a substantially pure population of isolated adult cardiac derived progenitor cells of the present invention, is administered to a subject having myocardial infarction. It is envisioned that once administered, the cells differentiate into at least one cardiac cell type selected from the group consisting of myocytes, endothelial cells, vascular smooth muscle cells, and fibroblasts. It is contemplated that the differentiated cells can alleviate the symptoms associated with myocardial infarction. For example, the injected cells will migrate to the infarcted myocardium. The migrated isolated adult cardiac derived progenitor cells of the present invention then differentiate into myocytes. The myocytes assemble into myocardium tissue resulting in repair or regeneration of the infarcted myocardium.

Further embodiments of the present invention involve a method of targeting injured myocardium by administering to a subject an isolated adult cardiac derived progenitor cell of the present invention or a substantially pure population of isolated adult cardiac derived progenitor cells of the present invention, wherein the cells migrate or home and attach to the injured myocardium. The isolated adult cardiac derived progenitor cell of the present invention are administered intravenously to the subject. Thus, the cells maneuver the systemic circulation and migrate or target or home to the damaged or injured myocardium. Once the isolated adult cardiac derived progenitor cell of the present invention have migrated to the damaged myocardium, the cells differentiate into myocytes, smooth muscle cells or endothelial cells. It is well known to those of skill in the art that these cell types are essential to restore both structural and functional integrity to a damaged myocardium. Thus, targeting the myocardium with the isolated adult cardiac derived progenitor cell of the present invention or a substantially pure population of isolated adult cardiac derived progenitor cells of the present invention results in repair of a damaged myocardium.

In further embodiments, the present invention involves a method of repairing injured coronary vessels by administering to the subject an effective amount of isolated adult cardiac derived progenitor cell of the present invention such that the amount results in regeneration of coronary vascular cells to repair the coronary vasculature.

Another embodiment of the present invention comprises a method of treating heart failure in a subject comprising the step of administering to the subject an effective amount of an adult cardiac-derived progenitor cell of the invention. In this aspect, the cell expresses at least one of the following: a detectable amount of Abcg2 protein or an mRNA encoding Abcg2 (Abcg2^{pos}); a detectable amount of MDR1 protein or an mRNA encoding MDR1 (MDR1^{pos}); a detectable amount of Mef2a protein or an mRNA encoding Mef2A (Mef2^{pos}); a detectable amount of Gata4 protein or an mRNA encoding Gata4 (Gata4^{pos}); a detectable amount of dHAND protein or an mRNA encoding dHAND (dHAND^{pos}); a detectable amount of Tbx2 protein or an mRNA encoding Tbx2 (Tbx2^{pos}); a detectable amount of Tbx5 protein or an mRNA encoding Tbx5 (Tbx5^{pos}); a detectable amount of Tbx20 protein or an mRNA encoding Tbx20 (Tbx20^{pos}); a detectable amount of CD105 protein or an mRNA encoding CD105 (CD105^{pos}); a detectable amount of CD90 protein or an mRNA encoding CD90 (CD90^{pos}); and/or a detectable amount of CD44 protein or an mRNA encoding CD44 (CD44^{pos}). The selected cell does not express at least one of the following: a detectable amount of c-kit protein or an mRNA encoding c-kit (c-kit^{neg}); a detectable amount of Lin protein or an mRNA encoding Lin (Lin^{neg}); a detectable amount of Lmo2 protein or an mRNA encoding Lmo2 (Lmo2^{neg}); a detectable amount of Gata2 protein or an mRNA encoding Gata2 (Gata2^{neg}); a detectable amount of Tal protein or an mRNA encoding Tal (Tal^{neg}); a detectable amount of CD45 protein or an mRNA encoding CD45 (CD45^{neg}); a detectable amount of CD31 protein or an mRNA encoding CD31 (CD31^{neg}); and/or a detectable amount of CD34 protein or an mRNA encoding CD34 (CD34^{neg}), wherein the amount of cells administered to the subject is effective in at least partially restoring cardiac function. Heart failure can be considered essentially as a progressive disease of apoptotically-mediated cardiomyocyte loss that eventually results in an impaired functional capacity of the cardiac muscle. Thus, it is envisioned that administration of an adult cardiac-derived progenitor cell of the present invention to the subject may at least partially counteract the loss of cardiomyocytes due to apoptosis by replenishing or restoring the cardiomyocytes, leading to restoration of cardiac function. The restoration of cardiomyocytes may treat and/or minimize the heart failure suffered by the subject.

A further embodiment is a method of modulating the loss of cardiomyocytes in a subject comprising the step of administering to the subject an effective amount of an adult cardiac-derived progenitor cell of the invention. In this aspect, the cell expresses at least one of the following: a detectable amount of Abcg2 protein or an mRNA encoding Abcg2 (Abcg2^{pos}); a detectable amount of MDR1 protein or an mRNA encoding MDR1 (MDR1^{pos}); a detectable amount of Mef2a protein or an mRNA encoding Mef2A (Mef2a^{pos}); a detectable amount of Gata4 protein or an mRNA encoding Gata4 (Gata4^{pos}); a detectable amount of dHAND protein or an mRNA encoding dHAND (dHAND^{pos}); a detectable amount of Tbx2 protein or an mRNA encoding Tbx2 (Tbx2^{pos}); a detectable amount of Tbx5 protein or an mRNA encoding Tbx5 (Tbx5^{pos}); a detectable amount of Tbx20 protein or an mRNA encoding Tbx20 (Tbx20^{pos}); a detectable amount of CD105 protein or an mRNA encoding CD105 (CD105^{pos}); a detectable amount of CD90 protein or an mRNA encoding CD90 (CD90^{pos}); and/or a detectable amount of CD44 protein or an mRNA encoding CD44 (CD44^{pos}). The selected cell does not express at least one of the following: a detectable amount of c-kit protein or an mRNA encoding c-kit (c-kit^{neg}); a detectable amount of Lin protein or an mRNA encoding Lin (Lin^{neg}); a detectable amount of Lmo2 protein or an mRNA encoding Lmo2 (Lmo2^{neg}); a detectable amount of Gata2 protein or an mRNA encoding Gata2 (Gata2^{neg}); a detectable amount of Tal protein or an mRNA encoding Tal (Tal^{neg}); a detectable amount of CD45 protein or an mRNA encoding CD45 (CD45^{neg}); a detectable amount of CD31 protein or an mRNA encoding CD31 (CD31^{neg}); and/or a detectable amount of CD34 protein or an mRNA encoding CD34 (CD34^{neg}), wherein the amount of cells administered to the subject is effective in at least partially restoring cardiomyocytes. A loss of cardiomyocytes can be related to heart failure and apoptosis. It is envisioned that administration of the cells to a subject that has suffered a loss of cardiomyocytes may treat and/or prevent heart failure in the subject.

E. Cell Delivery

Accordingly, the invention involves the administration of adult cardiac-derived progenitor cell of the invention or a pharmaceutical composition of the present invention as a treatment or prevention of any one or more of these conditions or other conditions involving cardiovascular disease, as well as compositions for such treatment or prevention. It is envisioned one of skill in the art will know the most advantageous routes of administration depending upon the disease. In specific embodiments, it is contemplated that an adult cardiac-derived progenitor cell of the invention or pharmaceutical composition can be administered via injection, which includes, but is not limited to subcutaneous, intravenous, intra-arterial, intramuscular, intraperitoneal, intramyocardial, transendocardial, transepicardial, intranasal and intrathecal.

Introducing cells into cardiac tissue may be accomplished by any means known in the medical arts, including but not limited to grafting and injection. The cells can be introduced into myocardium with or without a natural or artificial support, matrix, or polymer. It should be understood that adult cardiac-derived progenitor cells can be injected or grafted into or at a site separate and/or apart from the diseased or damaged tissue and allowed to migrate to the site of injury.

By way of a none-limiting example, a therapeutic amount of human adult cardiac-derived progenitor cells cultured for 10-15 passages are filtered immediately before use through a 40 μm nylon mesh filter to remove cell aggregates and 10⁶ cells are injected in 100 μl of a pharmaceutical carrier. The cells may be injected via the right jugular vein, intracoronary delivery, transendocardial injection, or directly into ischemic tissue. Cells may be delivered once, or in sequential treatments. Other methods for delivery of cells to a patient, both known and heretofore unknown, are also included herein.

### EXPERIMENTAL EXAMPLES

The invention is further described in detail by reference to the following experimental examples. These examples are provided for purposes of illustration only, and are not intended to be limiting unless otherwise specified. Thus, the invention should in no way be construed as being limited to the following examples, but rather, should be construed to encompass any and all variations which become evident as a result of the teaching provided herein.

The materials and methods employed in the experiments disclosed herein and the results obtained are now described.

### Example 1

### Human Cardiac-Derived Cells are Obtained from Tissue by Explants or Cell Dissociation Methodology

### A. Materials and Methods:

### Tissue Donors

Two sources were established for the procurement of human heart tissue: The National Disease Research Interchange (NDRI) and Methodist Hospital (Houston, Tex.). Twenty-one (21) donors contributed to these studies; 17 from NDRI and 4 from Methodist Hospital. Donor characteristics are summarized in Table 1.

TABLE 1
Donor Characteristics

| | |
|---|---|
| Gender | 10 female; 11 male |
| Ethnic groups | 17 Caucasian; 3 African American; 1 Hispanic; |
| | 1 unknown |
| Age | 14, 21, 28, 28, 38, 39, 52, 54, 56, 57, 59, 59, 61, |
| | 61, 62, 63, 66, 69, 71, 77, 84 |
| Time from cross | 12, 12.5, 12.5, 13, 14, 15, 17.5, 17.5, 18, 18.5, 20, |
| clamp-time | 20, 20, 21, 23, 25, 26, average = 17.9 |
| processing (NDRI) | |

### Explant Method

Tissue sample of ˜1 gram were derived from atria or ventricular biopsy specimens-and stored in cell culture media at 4° C. Isolated myocardial tissue was cut into 1- to 2-mm³ pieces, washed with Ca²⁺-, and Mg²⁺-free phosphate buffered solution (PBS; Invitrogen), and digested three times for 5 minutes at 37° C. with 0.2% trypsin (Invitrogen) and 0.1% collagenase IV (Sigma, St. Louis, Mo.). The obtained cells were discarded, and the remaining tissue fragments washed with complete explant medium (CEM) (Iscove's Modified Dulbecco's Medium [IMDM] supplemented with 10% fetal calf serum, 100 U/mL penicillin G, 100 μg/mL streptomycin, 2 mmol/L L-glutamine, and 0.1 mmol/L 2-mercaptoethanol) were cultured as explants in CEM at 37° C. and 5% CO₂. After a period ranging from 1 (embryo) to 3 (adult) weeks, a layer of fibroblast-like cells was generated from adherent explants over which small, phase-bright cells migrated. These phase-bright cells were collected by pooling two washes with Ca²⁺—Mg²⁺-free PBS, one wash with 0.53 mmol/L EDTA (Versene, Invitrogen) (1 to 2 minutes), and one wash with 0.5 g/L trypsin and 0.53 mmol/L EDTA (Invitrogen) (2 to 3 minutes) at room temperature under visual control. The cells obtained (from 10⁴ to 4×10⁵ cells/explant) were seeded at ≈0.5 to 2×10⁵ cells/mL in collagen coated multiwell plates (BD Biosciences) in cardiosphere-growing medium (CGM) (35% complete IMDM/65% DMEM-Ham F-12 mix containing 2% B27, 0.1 mmol/L 2-mercaptoethanol, 10 ng/mL epidermal growth factor [EGF], 20 ng/mL basic fibroblast growth factor [bFGF], 40 nmol/L cardiotrophin-1, 40 nmol/L thrombin, antibiotics, and L-Glu, as in CEM). Isolation of the cardiosphere-forming cells could be performed at least 4 times at 6- to 10-day intervals from the same explant. Cardiospheres (CSs) were passaged every 2 to 3 days by partial changing of the medium and mechanical trituration of the larger clusters.

### Cell Dissociation Methodology

Tissue sample of ˜1 gram were derived from atria or ventricular biopsy specimens as described above and stored in cell culture media at 4° C. . Tissue is finely minced and incubated in a cocktail of enzymes (Blendzyme/DNAse). A percoll gradient is used to eliminate unwanted cells and debris. Cells are plated onto collagen or poly-D lysine coated dishes and allowed to grown until they are ready for expansion and analysis.

### Quantitative Reverse Transcriptase Polymerase Chain Reaction (QRT-PCR) Differentiation Assays

For real time QRT-PCR differentiation assays, cells are plated in 96-well plated at a density of 5-10,000 cells per well. RNA is isolated from 5,000 cells using the Tripure Isolation Kit (Roche Applied Science) and subjected to two rounds of amplification.

### B. Results:

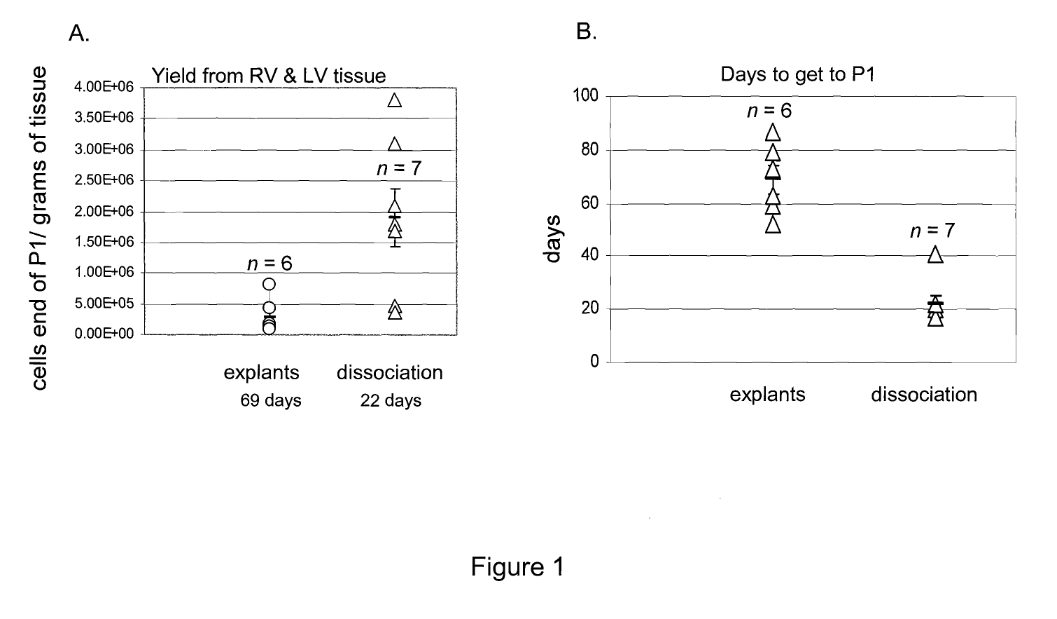

Two modes of generating dissociated human cardiac-derived cells were compared, explants vs. cell dissociation. The cell dissociation method uses enzymatic digestion of minced tissue fragments into a single cell suspension using a cocktail of enzymes. The cells are purified on a Percoll gradient and plated. The explant method uses a milder mix of enzymes (trypsin and collagenase IV) to release loose cells such as erythrocytes and leukocytes. The released cells are discarded and the tissue fragments are put into medium. Spontaneously outgrowing cells are later harvested. The cell dissociation method gave approximately seven (7) times more cells per gram of cardiac tissue (FIG. 1A) in one third of the time (FIG. 1B) than the explant method.

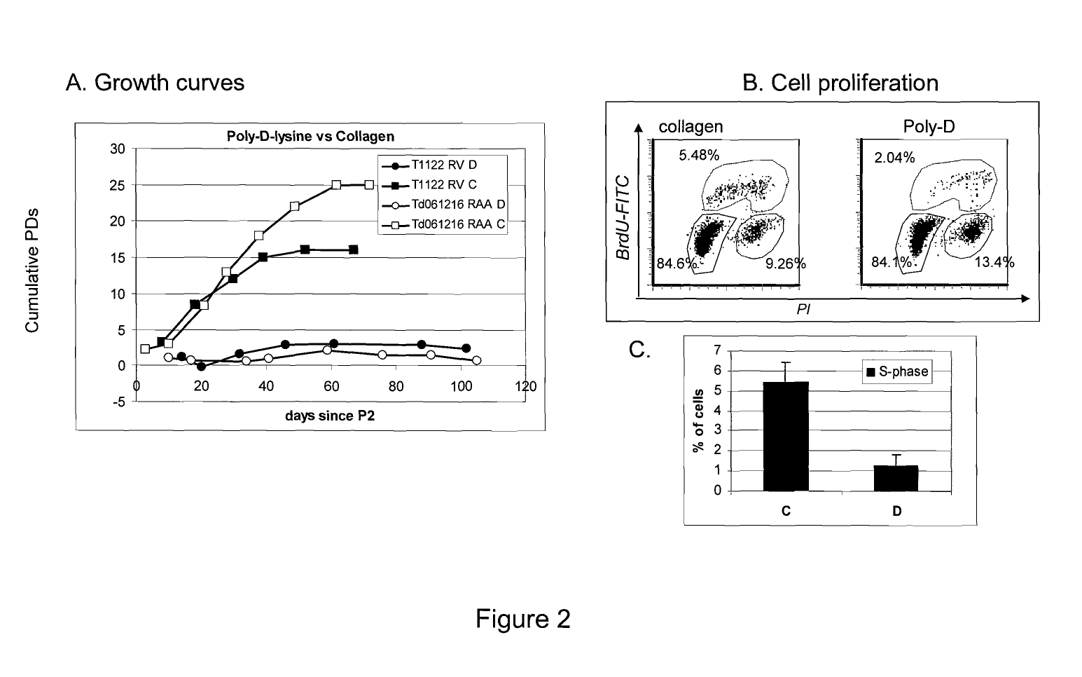

Propagation of human cardiac-derived cells was done on either collagen- or poly-D lysine coated dishes. A comparison of these conditions is depicted in FIG. 2. Human cardiac-derived cells grown on collagen-coated dishes exhibit more robust proliferation than cells obtained from the same samples but grown on poly-D lysine coated dishes (FIG. 2A). In addition, a larger percentage of cells were observed to be in sS-phase when grown on collagen coated dishes (FIG. 2B).

### Example 2

### Characterization of Human-Derived Cardiac Cells Obtained Using the Dissociation Method

### A. Materials and Methods

RT-PCR was performed on human derived cardiac cells obtained using the dissociation methods described above and Superscript One step RT-PCR from Invitrogen. Reverse transcription occurs at 48° C. for 30 minutes, denaturing for 2 minutes at 94° C. is followed by 40 cycles of 94° C. for 15 seconds, annealing at 55° C. (or as indicated) for 30 seconds, elongation at 72° C. for 30 seconds, the protocol ends with 10 minutes at 72° C.

### B. Results

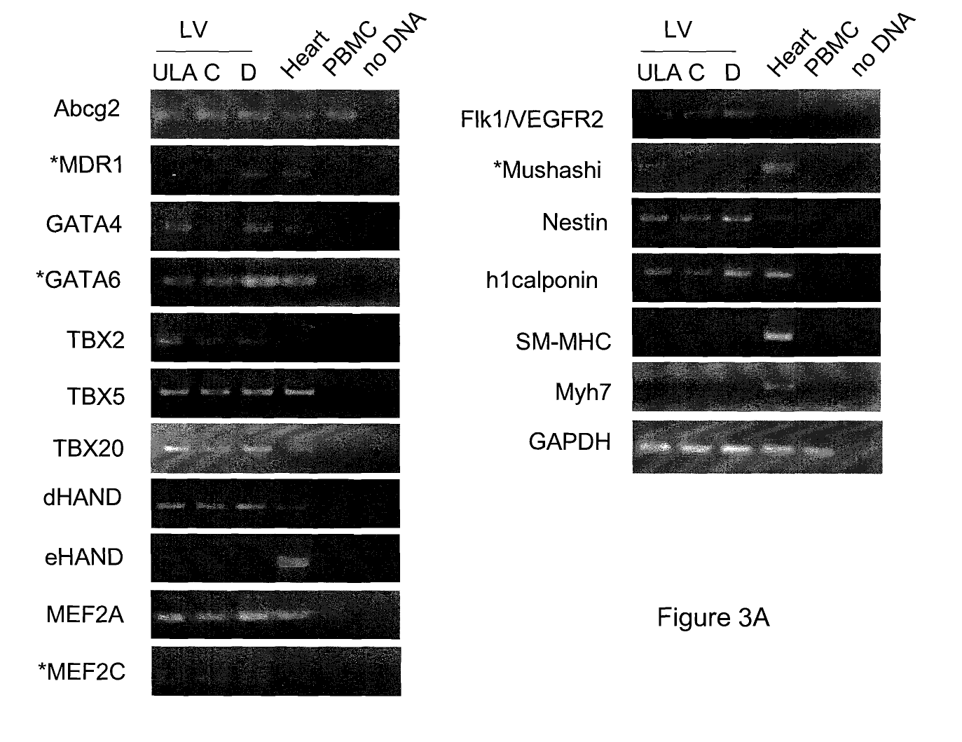

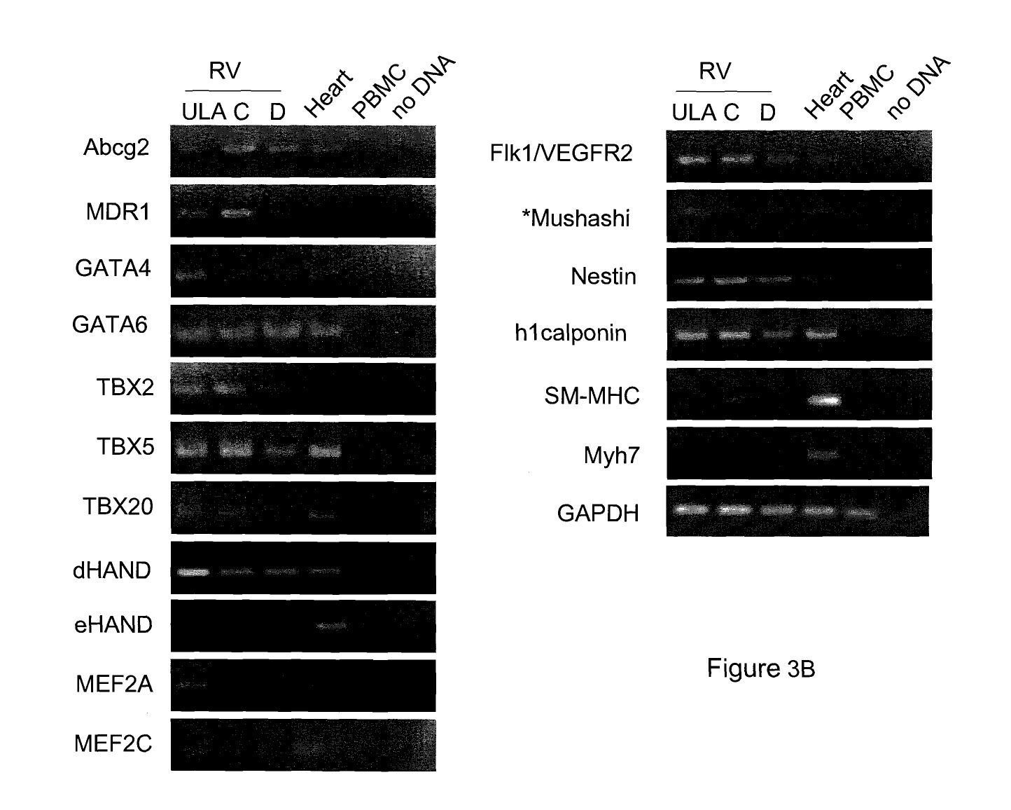

Human derived cardiac cells obtained by the cell dissociation method express a variety of markers that can be used in the isolation, purification, and selection of the cell. FIGS. 3A and 3B depict the results of RT-PCR performed on cells obtained from the left and right ventricles, respectively. RNA obtained from cells grown on ultra low attachment plates, collagen or poly lysine coated plates was probed for a variety of potential biomarkers. Tissue was obtained from the left ventricle (LV). ULA=ultra low attachment; C=collagen; D=poly-D lysine; Heart; PBMC=peripheral blood mononuclear cells. Negative controls were run without DNA template.

### Example 3

### Side Population (SP) as a Candidate for Cardiac Stem/Progenitor Cells

SP cells are identified based on their unique capacity to efflux the DNA-binding dye Hoechst 33342 (Goodell, 1997, Nature Medicine 3:1337-1345). SP cells are enriched for long-term self-renewal and multipotency in various tissues. In these experiments, SP cells are identified in human cardiac-derived cells derived from right atria appendix (RAA), right atria (RA), left atrial appendix (LAA), right ventricle (RV), and left ventricle (LV) obtained from 21 donors.

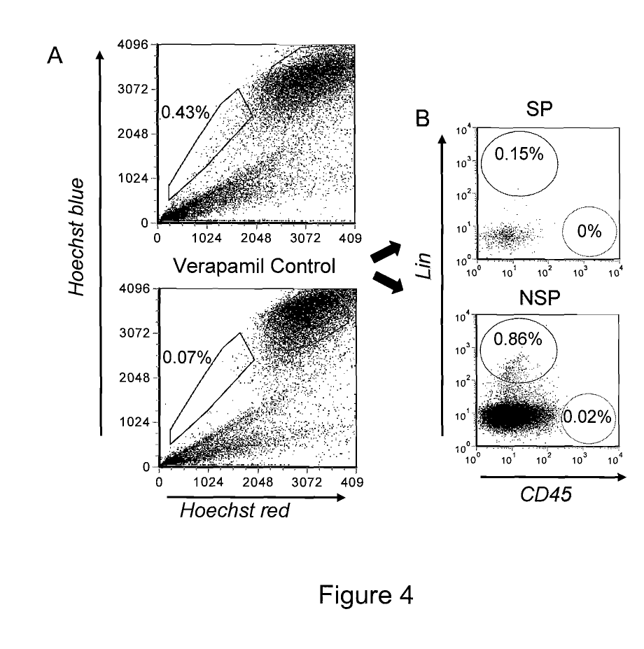

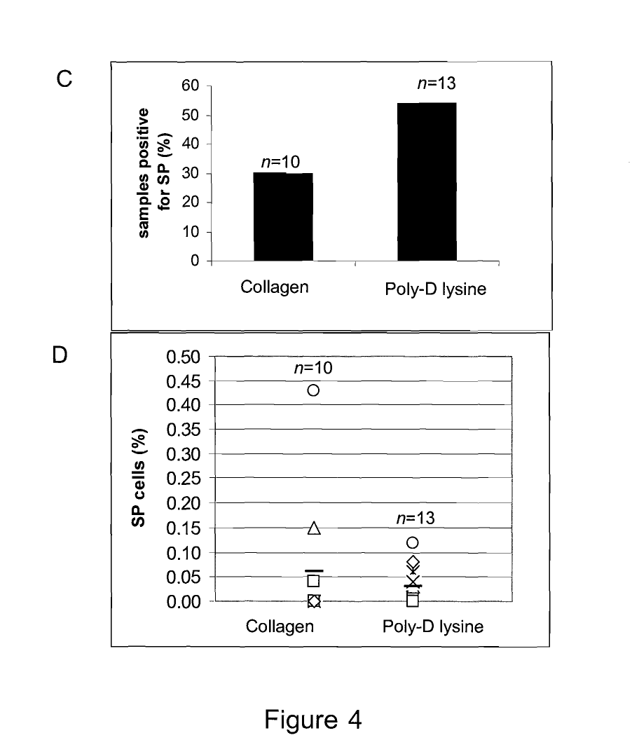

Human cardiac-derived cells were analyzed by flow cytometry according to their ability to efflux Hoechst dye 33342 to identify side population (SP) cells and non-SP (NSP) cells (FIG. 4A and FIG. 4B). As a control, cells were treated with Verapamil (an inhibitor of Hoechst extrusion). Treatment with Verapamil resulted in the expected disappearance of the SP cells. Human cardiac-derived SP cells are substantially negative for expression of hematopoietic progenitor marker CD45 and for antigens recognized by the hematopoietic lineage marker cocktail (Lin). NSP cells exhibit low levels of Lin+ cells (FIG. 4B). The percent of samples positive for SP cells when human cardiac-derived cells is related to whether cells were grown on either collagen (C) or poly-D lysine (D) (FIG. 4C). FIG. 4D is a graph depicting the per cent SP cells present when human cardiac-derived cells were grown on either collagen (C) or poly-D lysine (D).

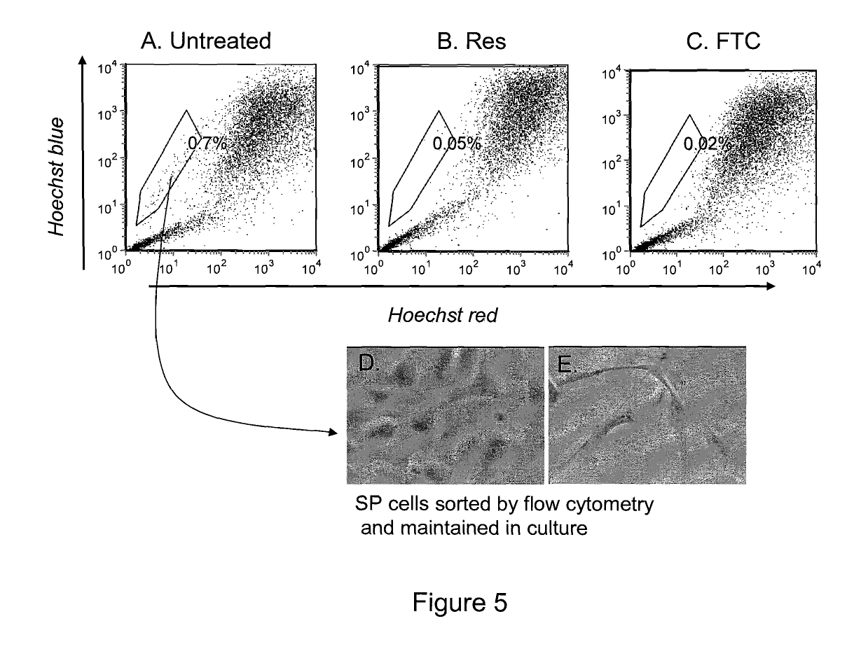

SP cells in human cardiac-derived cultures can be identified using different types of inhibitors. FIG. 5A through FIG. 5C depict two-color flow cytometry indicating human cardiac SP cells (region encompassed by solid polygon), by Hoechst dye exclusion. Untreated SP cells (controls) are depicted in FIG. 5A. Application of the pump inhibitor Resperpine to SP cells (Res; FIG. 5B), results in the loss of Hoechst dye exclusion in cells. Similarly, cells treated with another pump inhibitor, Fumitremorgin C (FTC) also exhibit a loss of Hoechst dye exclusion (FIG. 5C). Cells sorted by flow cytometry were subsequently maintained in culture (FIG. 5D and FIG. 5E).

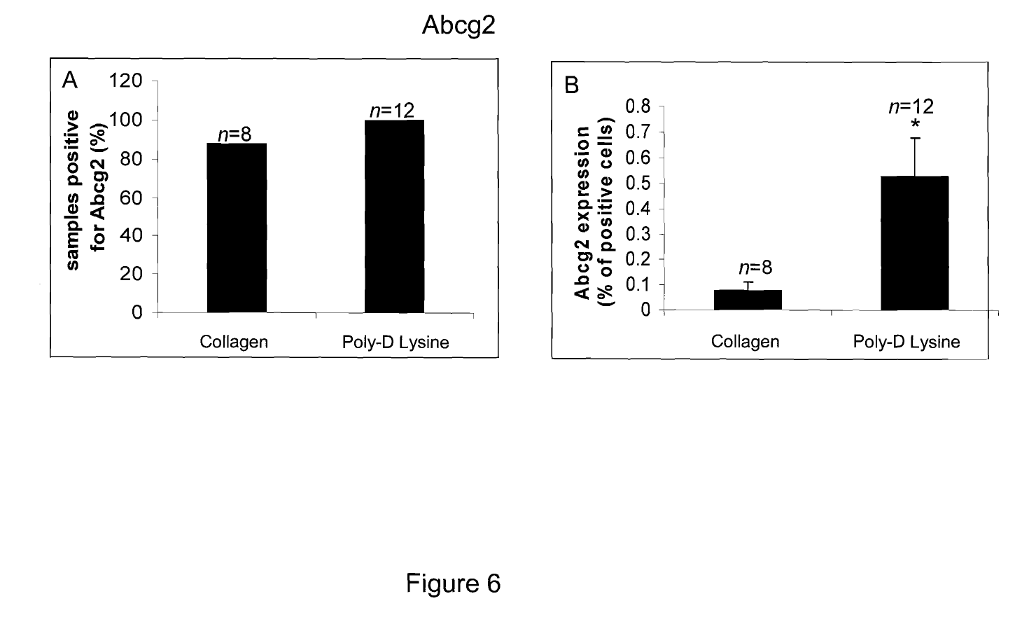

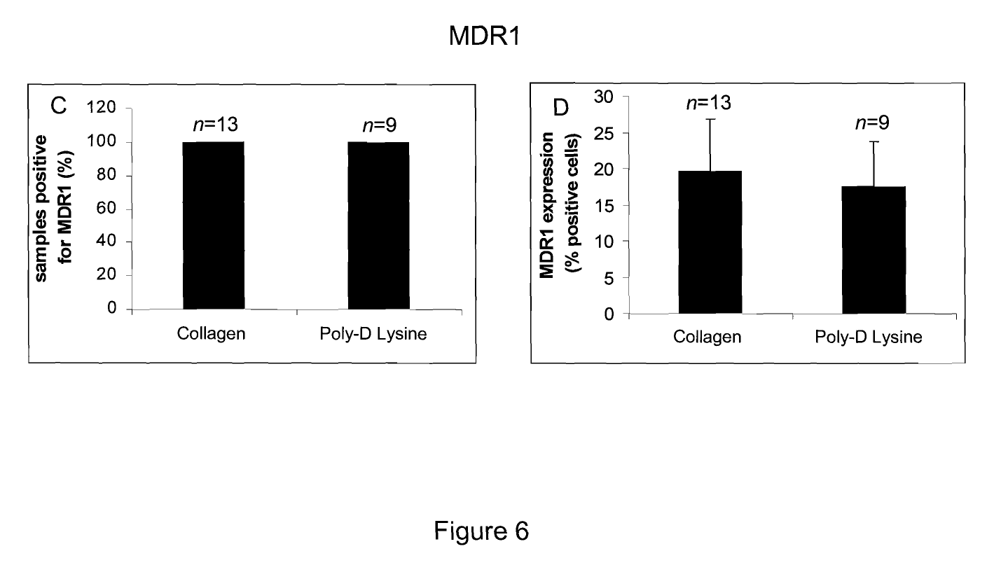

Expression of extrusion pumps such as Abcg2 and MDR1 were identified as putative molecular determinants of cells with the cardiac SP phenotype. Explant cultures were derived from cardiac regions including RAA, RA, LAA, RV. For MDR1 expression, cells were also derived from IVS and LV. The percent of samples positive for Abcg2 expression in cardiac derived cells and the percentage of cardiac derived cells that expressed Abcg2 are shown in FIG. 6A and FIG. 6B, respectively, for cells grown on collagen (C) or poly-D lysine (D). The percent of samples positive for MDR1 expression in cardiac derived cells and the percentage of cardiac derived cells that expressed MDR1 are shown in FIG. 6C and FIG. 6D, respectively, for cells grown on collagen (C) or poly-D lysine (D).

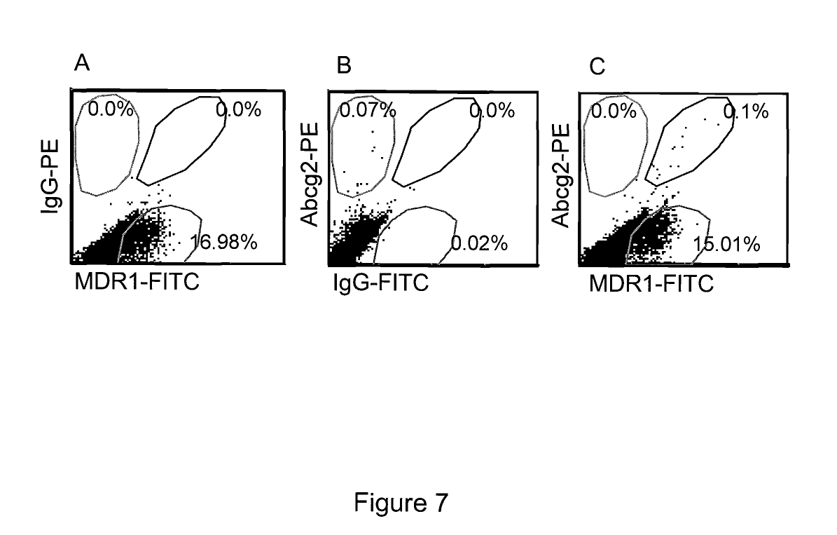

Two-color flow cytometry was used to determine the percentage of cells that were Abcg2+/MDR1−; Abcg2−/MDR1+; and Abcg2+/MDR1+ in cells derived from human cardiac explants cultured of the LV. FIGS. 7A depicts the separation of MDR1+ cells (16.98%). Isotype-specific irrelevant control antibodies (IgG-PE) were used in lieu of Abcg2. FIG. 7B depicts the separation of Abcg2 + cells (0.07%). Isotype-specific irrelevant control antibodies (IgG-FITC) were used in lieu of MDR1. FIG. 7C depicts the separation of Abcg2+/MDR+ SP cells (0.1%) and Abcg2−/MDR1+ SP ells (15.01%). No Abcg2+/MDR1− cells were identified in these samples.

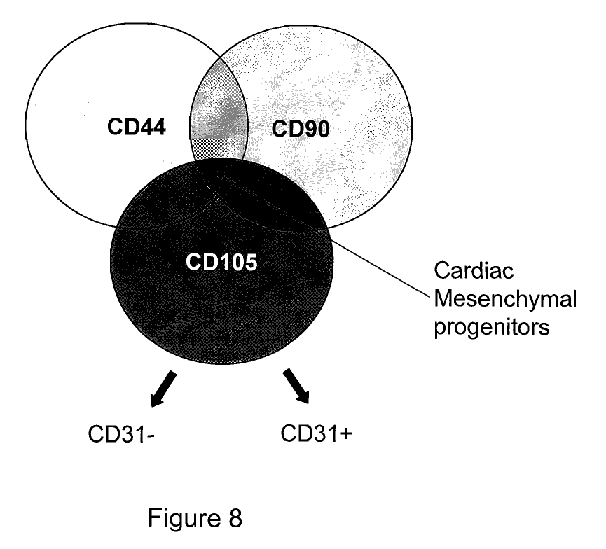

As a complementary approach, expression of markers that have been used to define mesenchymal stem cells from various sources were tested. The characterization of human cardiac-derived cells using mesenchymal stem-cell markers to define sub-populations of cardiac progenitor cells is summarized in FIG. 8. CD31−, but not CD31+ cardiac SP cells exhibit functional cardiomyogenic differentiation. CD31 can be co-expressed with CD105 and CD44 on endothelial cells. Also, mesenchymal stem cells from the hematopoietic system and other organs are by definition CD31−.

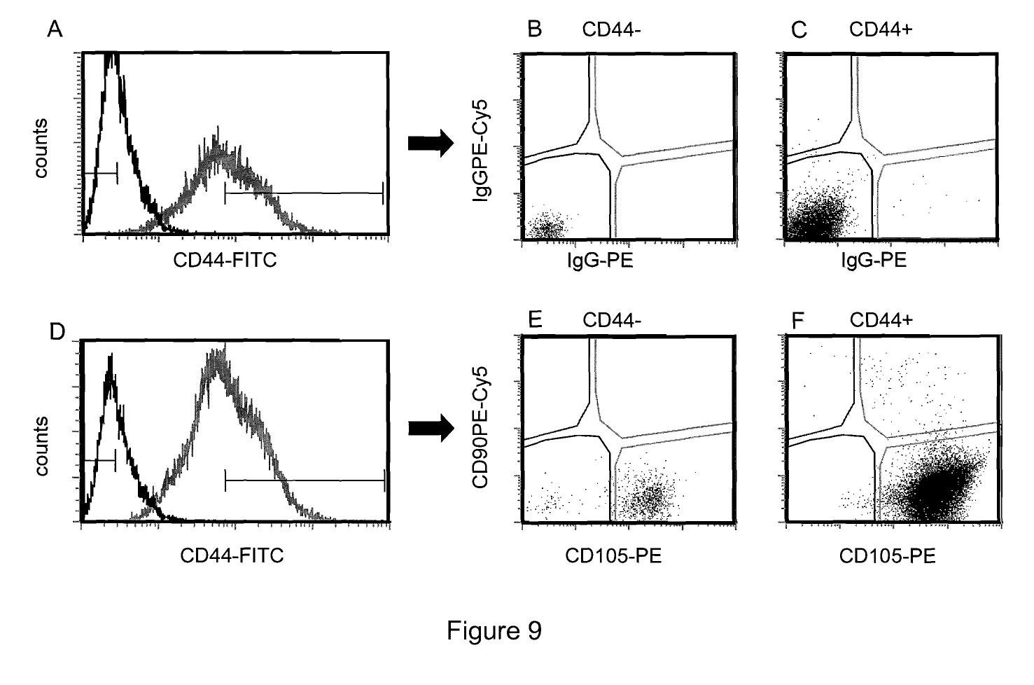

Three-color flow cytometry was performed on human cardiac derived cells to determine expression of mesenchymal markers CD44, CD90, and CD 105. FIGS. 9A and 9D are histograms comparing CD44 expression (grey) in cardiac derived cells to the isotype control (black). Dot blots gated on CD44− populations to show isotype controls and CD44+ populations to show isotype controls are shown in FIG. 9B and FIG. 9C respectively. Dot blot gated on CD44− populations and CD44+ populations to show CD90/CD105 staining are shown in FIG. 9E and FIG. 9F.

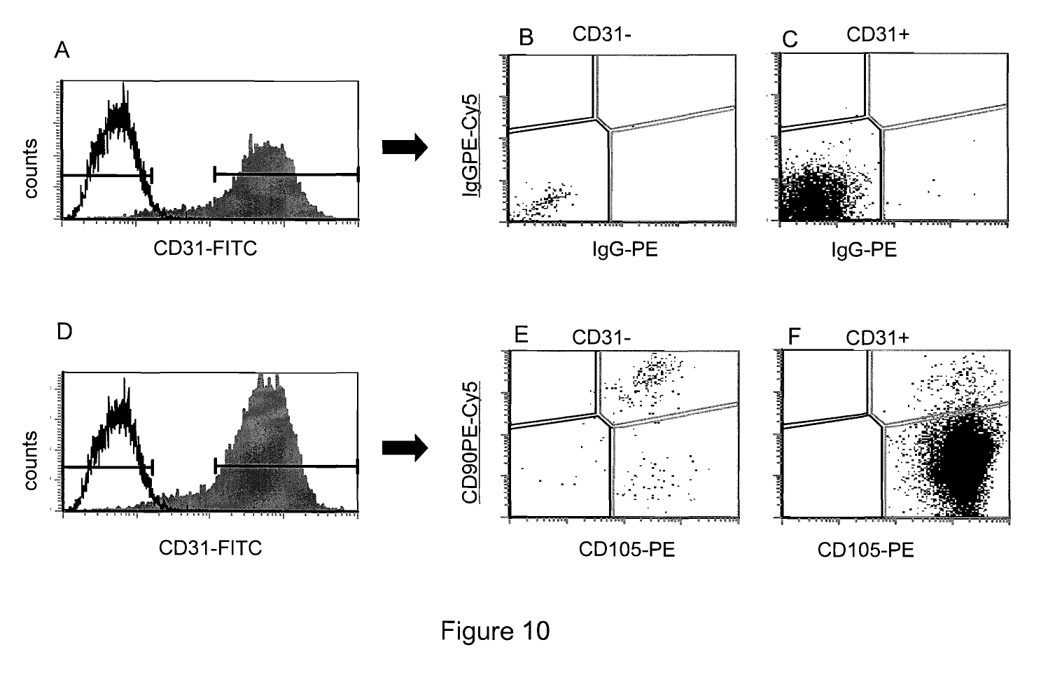

Three-color flow cytometry was performed on human derived cardiac cells to determine expression of mesenchymal markers CD31, CD90, and CD 105. Histograms comparing CD31 expression (grey) and profiles of isotype controls (black) are depicted in FIG. 10A and FIG. 10D). Dot blots gated on CD31− populations and CD31+ populations to show isotype controls are shown in FIG. 10B and FIG. 10C. Dot blots gated on CD31− populations and CD31+ populations to show CD90/CD105 staining are shown in FIG. 10E and FIG. 10F.

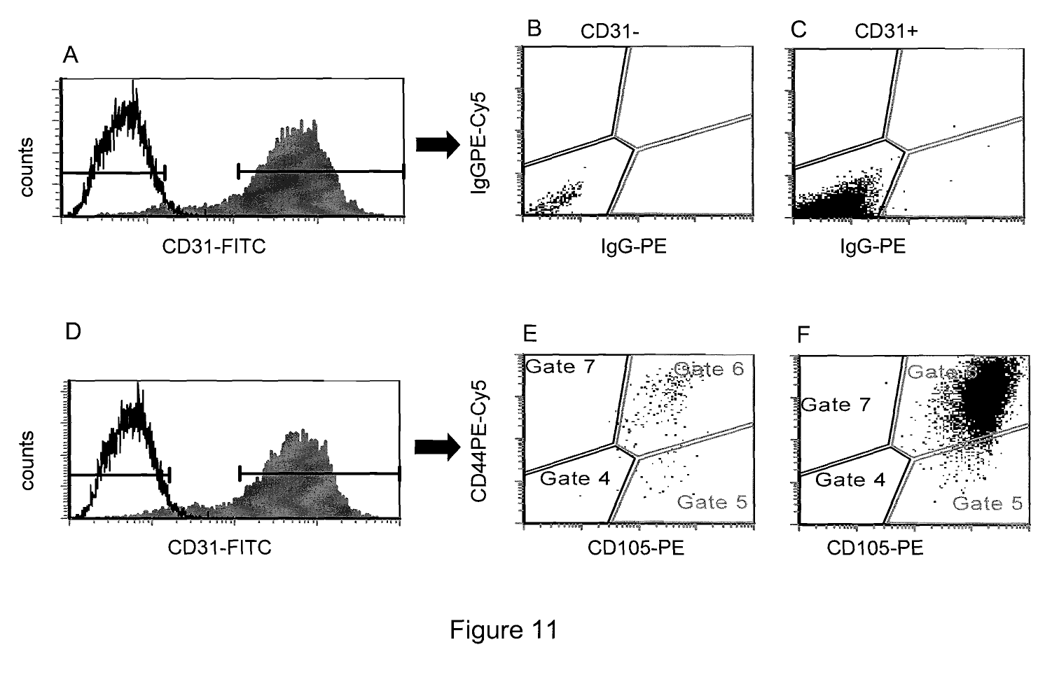

Three-color flow cytometry was performed on human derived cardiac cells to determine expression of mesenchymal markers CD31, CD44, CD105. Histograms depicting expression of CD31 (grey) and isotype control profiles (black) are shown in FIG. 11A and FIG. 11D. Dot blots gated on CD31− populations and CD31+ populations to show isotype controls are shown in FIG. 11B and FIG. 11C. Dot blots gated on CD31− populations and CD31+ populations to show CD44/CD105 staining are shown in FIG. 11E and FIG. 11F.

### Example 4

### Engraftment of Human Derived Cardiac Cells

### A. Materials and Methods

Animals: Female mice of SCID/NOD background (Jackson Lab) underwent left anterior coronary (LAD) ligation under general anesthesia at 8 weeks age.

Infarction and Cell Injection: After anesthesia induction in 5% Isoflurane chamber, tracheal intubation was done and mice were connected to a ventilator (Harvard apparatus). Isoflurane delivered through a vaporizer was kept at 1-2% throughout the procedure. Continuous ECG recording and temperature were monitored. Heart rate was kept above 400 during surgery. After anterior thoracotomy, LAD was localized and using propylene 7:00 suture, permanent ligation was achieved about 1 mm below left atrial edge. Infarction was confirmed with blanching of myocardium and ST elevation in the ECG.

Three injections were delivered intramyocardially using a 30 gauge needle in septal, apical and LV free wall sides of the blanching. Each injection comprised 10 μl of phosphate buffered saline (PBS) containing about 10,000 Abcg+ cells or Abcg− adult human cardiac derived progenitor cells.

The chest wall was closed using 5:00 polysorb suture and mice recovered from anesthesia. Buprenorphine SC (0.3 mg/kg) was administered before surgery and 6 h after surgery for pain control.

Histology Mice were euthanized 48 hours after cell injection by nucal extraction. Hearts were immediately harvested and weighed. The aorta was cannulized and hearts perfused with KCL (10 mM) and 10% Formalin for 10 minutes. Hearts were then fixed overnight in 10% Formalin. Hearts were sectioned the following day into lmm sections and sent to histology lab for slide preparation and paraffin molding.

The following probes were used: Alu probe hybridization was done on all samples to detect the presence of human DNA in mouse tissue. NKx2.5 and GATA-4 are both cardiac specific transcription factors that are early markers of precardiac cells, and consequently were used to detect the early stages of cardiac differentiation of the isolated adult cardiac derived progenitor cells that were injected into the cardiac myocardium. Connexin 43 is a connexin gap junction found in the heart and brain. Alpha sarcomeric actinin (a-SA) is a cardiac specific protein that stains Z lines and dots in stress fibers of myotubes in skeletal and cardiac muscle but not in non-sarcomeric muscle elements.

Positive control samples were obtained from human heart. Negative controls were obtained from an SCID/NOD mouse which was administered PBS injections without cells following infarction.

### Echocardiography

Echocardiograms were performed one day prior to and 48 hours post myocardial infarction using Vevo770 system long axis and short axis views, two dimensional (2D), and M-mode views of the heart obtained for mice under general anesthesia. Two mice received an intramyocardial injection as described elsewhere herein of Abcg2+ human cardiac derived progenitor cells, one mouse received an injection of Abcg2− human cardiac derived progenitor cells, and one mouse was injected with PBS. Mice were anesthetized in 5% Isoflurane chamber and anesthesia was maintained in 1-2% Isoflurane delivered via face masc. Temperature, ECG, respiratory and heart rates were monitored during the procedure. Images were obtained at heart rates above 450 beats per minute (bpm). Images were analyzed with Vevo 770 software and ejection fraction, left ventricular dimensions and wall thickness were determined.

### B. Results

### Histology

Positive control samples from human heart demonstrated staining for Alu, Nkx2.5, GATA4, Cx43, and a-SA. Negative control samples of cardiac tissue were obtained from an SCID/NOD mouse that received intramyocardial injections of PBS post infarction. Tissue obtained from the negative control animal exhibited only staining for CX43 and a-SA, but not Alu.

Cardiac tissue obtained from SCID/NOD mice injected post-infarction with Abcg2+ human cardiac derived progenitor cells demonstrated the presence of Alu+ cells alone and in clusters, demonstrating the engraftment of the precursor cells in the mouse hearts. Alu+ cells co=expressed Nkx2.5 and a-SA in some cases. These data indicate that cardiac engraftment and differentiation of the progenitor cells had begun in the heart.

Cardiac tissue obtained from SCID/NOD mouse injected post-infraction with Abcg2− human cardiac derived progenitor cells demonstrated Alu staining, but not co-staining for either Nkx2.5 or GATA-4.

### Echocardiograph

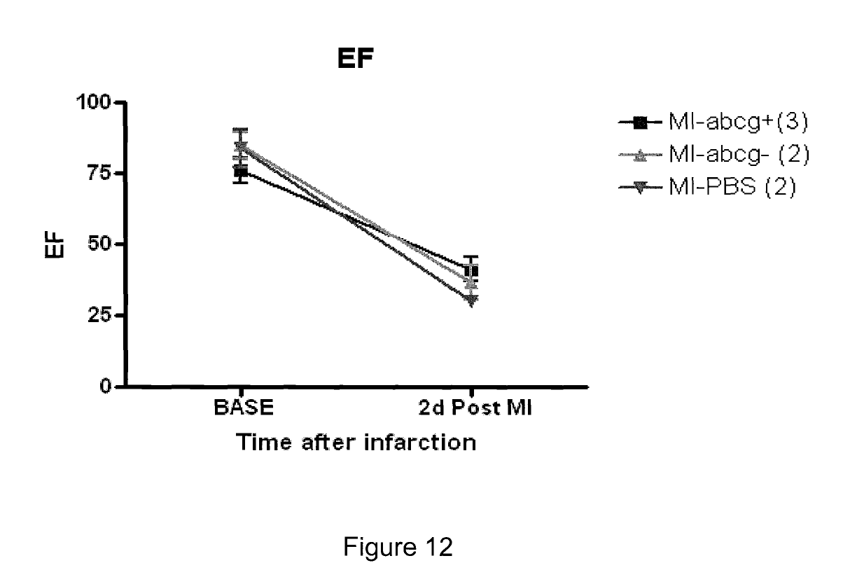

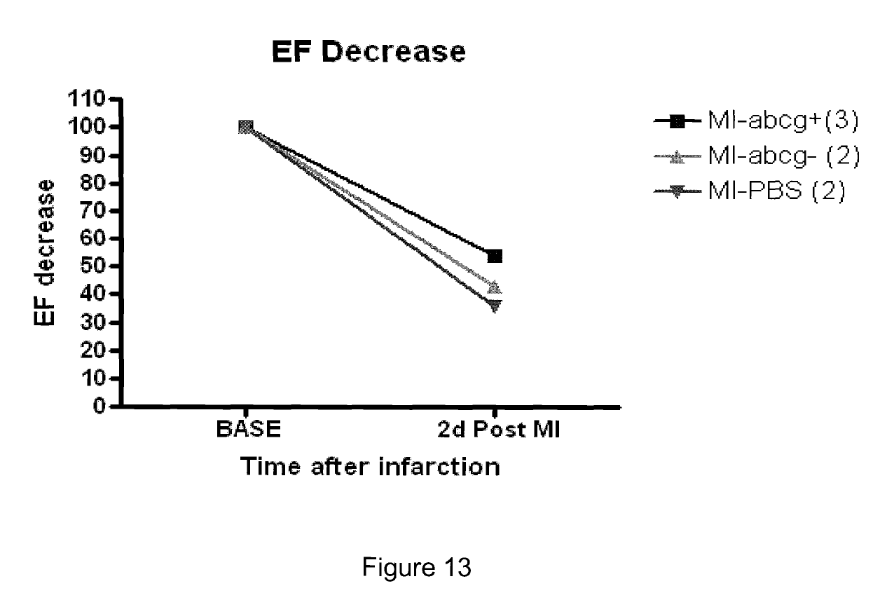

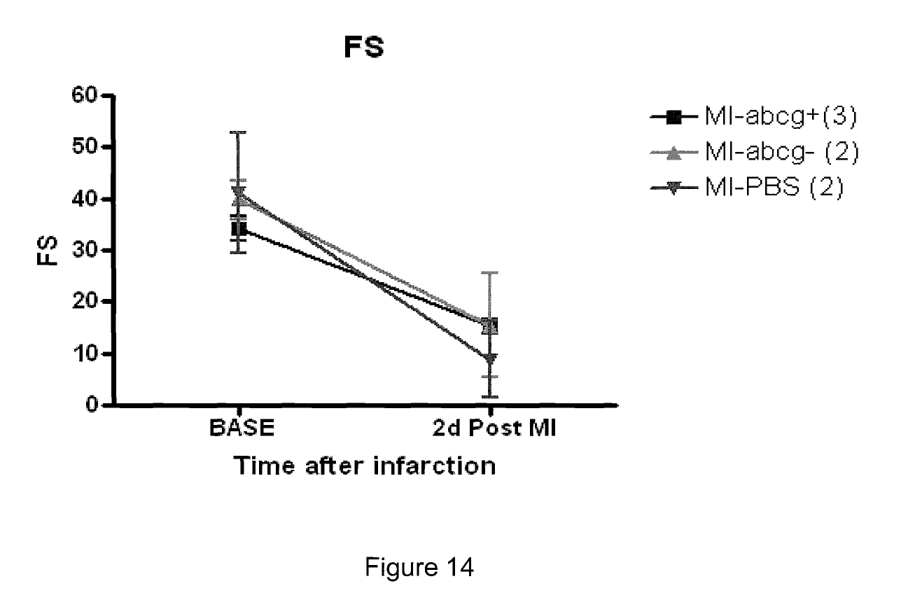

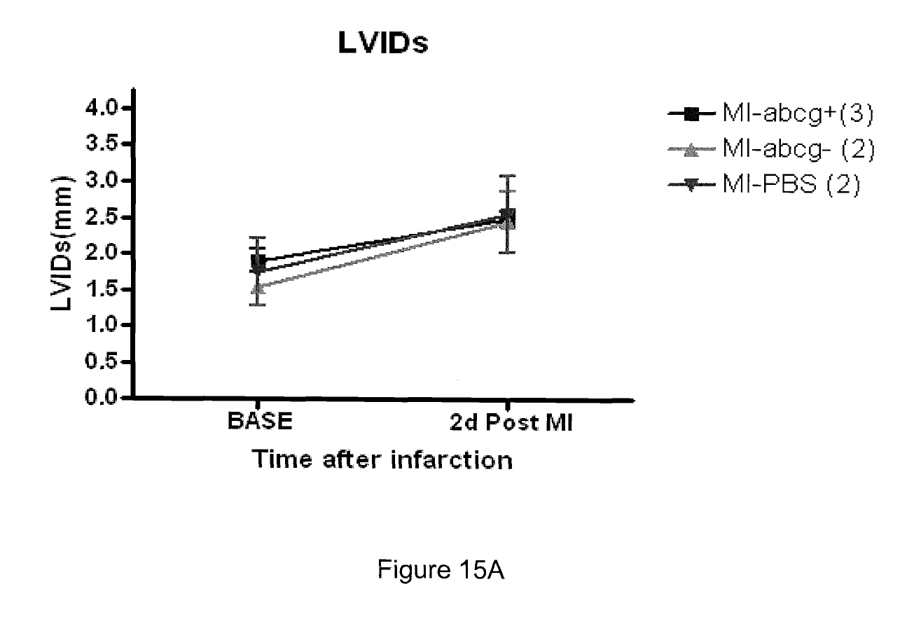

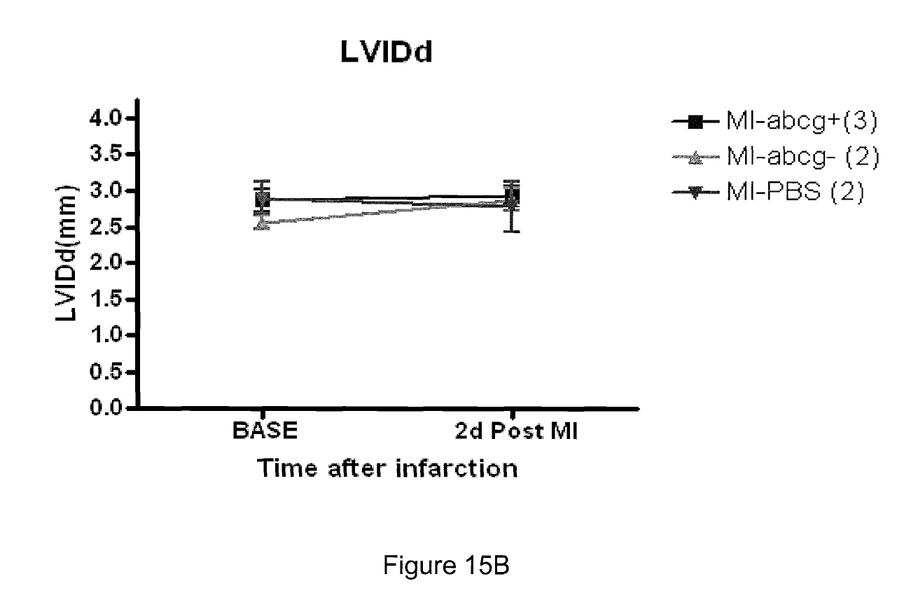

Echocardiographic and electrocardiogram (ECG) data were obtained on all mice pre- and 48 hours post-infarction. Ejection fraction and fractional shortening were both measured (FIG. 12, FIG. 13, and FIG. 14) and were reduced immediately after induced myocardial infarction. Accordingly, systolic dimension (LVIDS) increased due to decreased contractility of the myocardium (FIG. 15A). Diastolic dimension (LVIDd; FIG. 15B) did not changed during the time course of these experiments.

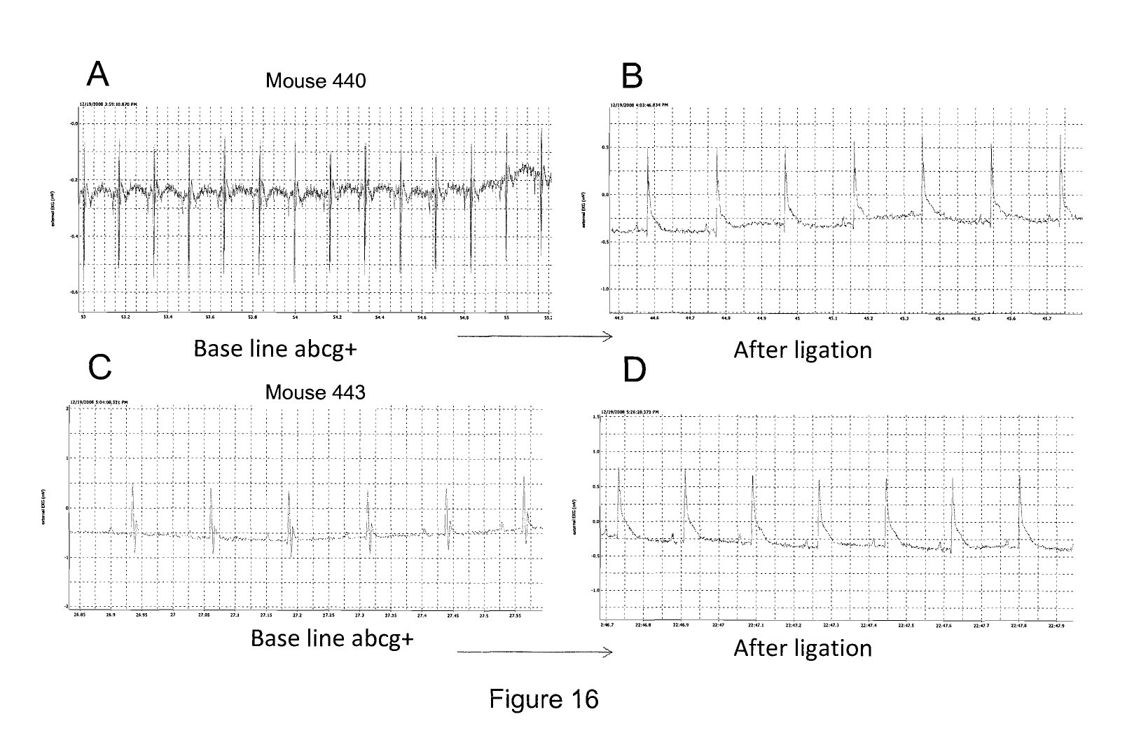

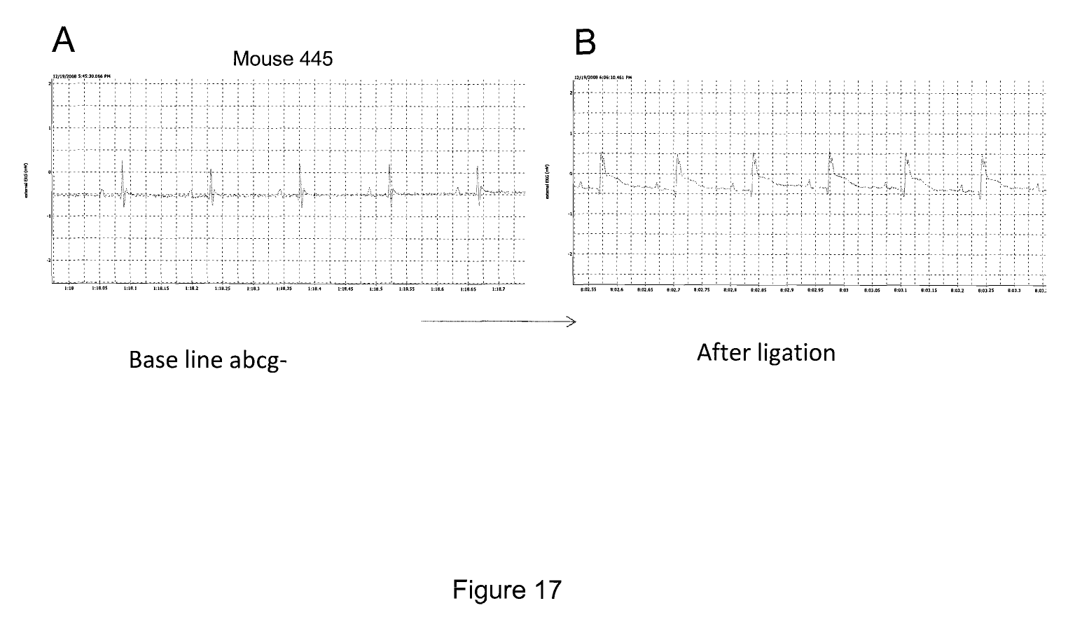

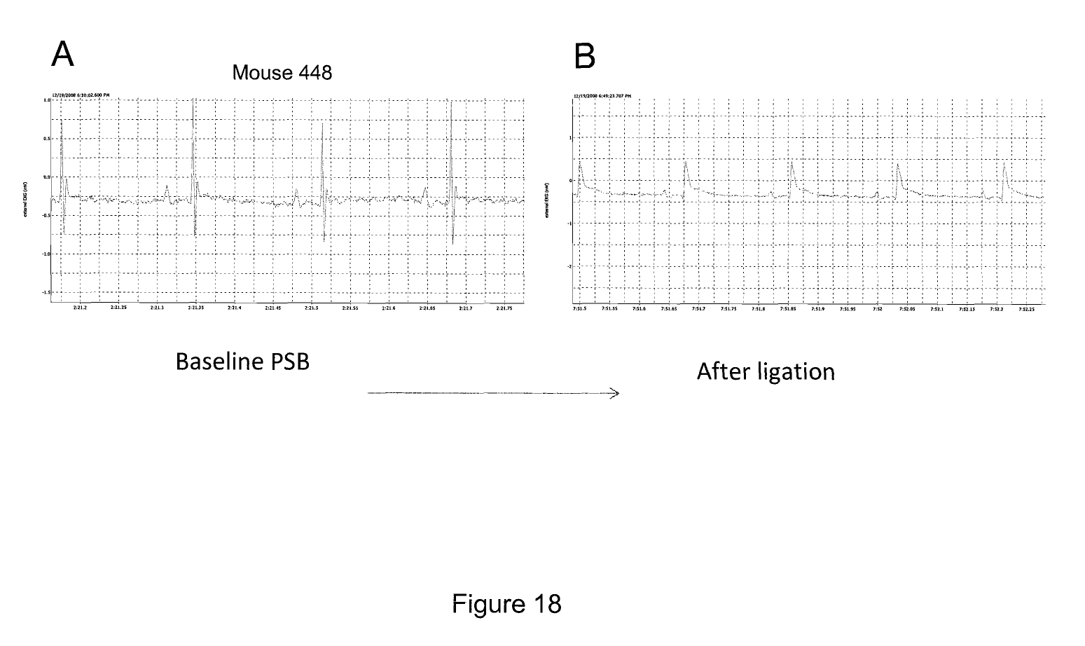

ECGs were obtained before and after ligation of the left anterior descending coronary artery (LAD) in mice that were injected with Abcg2+ and Abcg2− human cardiac derived progenitor cells (FIGS. 16 and 17) or PBS (FIG. 18). In all cases, the ECG demonstrated ST segment elevation consistent with infarction.

The disclosures of each and every patent, patent application, and publication cited herein are hereby incorporated herein by reference in their entirety.

While this invention has been disclosed with reference to specific embodiments, it is apparent that other embodiments and variations of this invention may be devised by others skilled in the art without departing from the true spirit and scope of the invention. The appended claims are intended to be construed to include all such embodiments and equivalent variations.

What is claimed:

1. An isolated human adult progenitor cell, wherein said cell is isolated from a cardiac tissue sample, wherein said cell expresses an Abcg2 protein or an mRNA encoding Abcg2, wherein said cell also expresses at least one cardiac transcription factor protein selected from the group consisting of dHAND, Gata4, Mef2a, Tbx2, Tbx5, Tbx20, or an mRNA encoding said at least one cardiac transcription factor, and further wherein, said cell does not substantially express a c-kit protein or an mRNA encoding c-kit at the time of cell isolation.

2. The cell of claim 1, wherein said cell further expresses a mesenchymal stem cell marker protein or a nucleic acid encoding said stem cell marker protein selected from the group consisting of CD105, CD90, and CD44.

3. The cell of claim 1, where said cell does not express at least one hematopoietic biomarker protein or a nucleic acid encoding said biomarker protein selected from the group consisting of CD45, CD34, CD31, Lin, Lmo2, Gata2, and Tal.

4. The cell of claim 1, wherein said cell is capable of differentiating into a cardiac muscle cell.

5. A pharmaceutical composition comprising a therapeutically effective amount of an isolated human adult progenitor cell, wherein said cell is isolated from a cardiac tissue sample, wherein said cell expresses an Abcg2 protein or an mRNA encoding Abcg2, wherein said cell also expresses at least one cardiac transcription factor protein selected from the group consisting of dHAND, Gata4, Mef2a, Tbx2, Tbx5, Tbx20, or an mRNA encoding said at least one cardiac transcription factor, and further wherein, said cell does not substantially express a c-kit protein or an mRNA encoding c-kit at the time of cell isolation.

6. The pharmaceutical composition of claim 5, wherein said cell further expresses a mesenchymal stem cell marker protein or a nucleic acid encoding said stem cell marker protein selected from the group consisting of CD105, CD90, and CD44.

7. The pharmaceutical composition of claim 5, where said cell does not express at least one hematopoietic biomarker protein or a nucleic acid encoding said biomarker protein selected from the group consisting of CD45, CD34, CD31, Lin, Lmo2, Gata2, and Tal.

8. The pharmaceutical composition of claim 5, wherein said cell is capable of differentiating into a cardiac muscle cell.

9. A cultured human adult progenitor cell, wherein said cell is isolated from a cardiac tissue sample, wherein said cell expresses an Abcg2 protein or an mRNA encoding Abcg2, wherein said cell also expresses at least one cardiac transcription factor protein selected from the group consisting of dHAND, Gata4, Mef2a, Tbx2, Tbx5, Tbx20, or an mRNA encoding said at least one cardiac transcription factor, and further wherein, said cell does not substantially express a c-kit protein or an mRNA encoding c-kit at the time of cell isolation.

10. The cell of claim 9, wherein said cell further expresses a mesenchymal stem cell marker protein or a nucleic acid encoding said stem cell marker protein selected from the group consisting of CD105, CD90, and CD44.

11. The cell of claim 9, where said cell does not express at least one hematopoietic biomarker protein or a nucleic acid encoding said biomarker protein selected from the group consisting of CD45, CD34, CD31, Lin, Lmo2, Gata2, and Tal.

12. The cell of claim 9, wherein said cell is capable of differentiating into a cardiac muscle cell.

13. A pharmaceutical composition comprising a therapeutic amount of a cultured human adult progenitor cell, wherein said cell is isolated from a cardiac tissue sample, wherein said cell expresses an Abcg2 protein or an mRNA encoding Abcg2, wherein said cell also expresses at least one cardiac transcription factor protein selected from the group consisting of dHAND, Gata4, Mef2a, Tbx2, Tbx5, Tbx20, or an mRNA encoding said at least one cardiac transcription factor, and further wherein, said cell does not substantially express a c-kit protein or an mRNA encoding c-kit at the time of cell isolation.

14. The pharmaceutical composition of claim 13, wherein said cell further expresses a mesenchymal stem cell marker protein or a nucleic acid encoding said stem cell marker protein selected from the group consisting of CD105, CD90, and CD44.

15. The pharmaceutical composition of claim 13, where said cell does not express at least one hematopoietic biomarker protein or a nucleic acid encoding said biomarker protein selected from the group consisting of CD45, CD34, CD31, Lin, Lmo2, Gata2, and Tal.

16. The pharmaceutical composition of claim 13, wherein said cell is capable of differentiating into a cardiac muscle cell.

17. A substantially pure population of adult human progenitor cells, wherein said population is derived from a cardiac tissue sample obtained from a human, wherein said population of cells is selected by fluorescence or magnetic cell sorting, and wherein said population of cells expresses an Abcg2 protein or an mRNA encoding Abcg2, wherein said population of cells also expresses at least one cardiac transcription factor protein selected from the group consisting of dHAND, Gata4, Mef2a, Tbx2, Tbx5, Tbx20, or an mRNA encoding said at least one cardiac transcription factor, and further wherein, said population of cells does not substantially express a c-kit protein or an mRNA encoding c-kit at the time of selection.

18. The population of adult human progenitor cells of claim 17, wherein said population of cells express at least one mesenchymal stem cell marker protein or a nucleic acid encoding said stem cell marker protein selected from the list consisting of CD105, CD90, and CD44.

19. The population of adult human progenitor cells of claim 17, where said population of cells does not express at least one hematopoietic biomarker protein or a nucleic acid encoding said biomarker protein selected from the group consisting of CD45, CD34, CD31, Lin, Lmo2, Gata2, and Tal.

20. The population of adult human cardiac-derived progenitor cells of claim 17, wherein said population of cells are capable of differentiating into cardiac muscle cells.

21. A composition comprising a therapeutic amount of an isolated adult human progenitor cell, wherein said cell is derived from a cardiac tissue sample obtained from a human, wherein said cell expresses an Abcg2 protein or an mRNA encoding Abcg2, wherein said cell also expresses at least one cardiac transcription factor protein selected from the group consisting of dHAND, Gata4, Mef2a, Tbx2, Tbx5, Tbx20, or an mRNA encoding said at least one cardiac transcription factor, and further wherein, said cell does not substantially express a c-kit protein or an mRNA encoding c-kit at the time of cell isolation.

22. The composition of claim 21, wherein said composition further comprises a pharmaceutical acceptable carrier.

23. The composition of claim 21, wherein said cell further expresses a mesenchymal stem cell marker protein or a nucleic acid encoding said stem cell marker protein selected from the group consisting of CD 105, CD90, and CD44.

24. The composition of claim 21, where said cell does not express at least one hematopoietic biomarker protein or a nucleic acid encoding said biomarker protein selected from the group consisting of CD45, CD34, CD31, Lin, Lmo2, Gata2, and Tal.

25. The pharmaceutical composition of claim 21, wherein said cell is capable of differentiating into a cardiac muscle cell.

26. A method of treating a patient with cardiovascular disease, said method comprising the steps of administering to said patient a therapeutically effective amount of a human progenitor cell, wherein said cell is isolated from a cardiac tissue sample, wherein when said cell is isolated, said cell expresses an Abcg2 protein or an mRNA encoding Abcg2, wherein said cell also expresses at least one cardiac transcription factor protein selected from the group consisting of dHAND, Gata4, Mef2a, Tbx2, Tbx5, Tbx20, or an mRNA encoding at said least one cardiac transcription factor, and further wherein, said cell does not substantially express a c-kit protein or an mRNA encoding c-kit at the time of cell isolation.

27. The method of claim 26, wherein said cell further expresses a mesenchymal stem cell marker protein or a nucleic acid encoding said stem cell marker protein selected from the group consisting of CD105, CD90, and CD44.

28. The method of claim 26, where said cell does not express at least one hematopoietic biomarker protein or a nucleic acid encoding said biomarker protein selected from the group consisting of CD45, CD34, CD31, Lin, Lmo2, Gata2, and Tal.

29. The method of claim 26, wherein said cardiovascular disease is selected from the list consisting of coronary artery disease, myocardial infarction, ischemic heart disease, and heart failure.

30. The method of claim 26, wherein said cell is admixed with a pharmaceutical acceptable carrier.

31. The method of claim 26, wherein said cell is autologous.

32. A method of treating damaged myocardium in a patient, said method comprising the steps of:
(a) obtaining cells from an adult cardiac tissue sample;
(b) selecting cells one or more times, before or after expanding said cells, to generate a substantially pure population of cardiac progenitor cells that are c-kit^{neg} and Abcg2^{pos} at the time of cell selection;
(c) expanding said cells to form a substantially pure population of adult human cardiac progenitor cells;
(d) administering a therapeutically effective amount of said cardiac progenitor cells to said patient.

33. A method of obtaining a population of human cardiac progenitor cells, said method comprising the steps of:
(a) obtaining cells from a cardiac tissue sample;
(b) selecting cells one or more times, before or after expanding said cells, using either fluorescent or magnetic cell sorting techniques, to generate a substantially pure population of cardiac progenitor cells, wherein said cells are substantially c-kit^{neg} and Abcg2^{pos} at the time of sorting;
(c) expanding said cells to form a substantially pure population of adult human cardiac progenitor cells.

34. The method of claim 33, wherein the cells are sorted at least once using one or more biomarkers.

35. An isolated human adult progenitor cell, wherein said cell is isolated from a cardiac tissue sample, wherein said cell expresses at least one mesenchymal stem cell marker protein or nucleic acid encoding said protein selected from the group consisting of CD105, CD90, and CD44, wherein said cell also expresses at least one cardiac transcription factor protein selected from the group consisting of dHAND, Gata4, Mef2a, Tbx2, Tbx5, Tbx20, or an mRNA encoding said at least one cardiac transcription factor, and further wherein, said cell does not substantially express a c-kit protein or an mRNA encoding c-kit at the time of cell isolation.

36. The cell of claim 35, wherein said cell further expresses an Abcg2 protein or an mRNA encoding Abcg2.

37. The cell of claim 35, where said cell does not express at least one hematopoietic biomarker protein or a nucleic acid encoding said biomarker protein selected from the group consisting of CD45, CD34, CD31, Lin, Lmo2, Gata2, and Tal.

38. The cell of claim 35, wherein said cell is capable of differentiating into a cardiac muscle cell.

39. A pharmaceutical composition comprising a therapeutically effective amount of an isolated human adult progenitor cell, wherein said cell is isolated from a cardiac tissue sample, wherein said cell expresses at least one mesenchymal stem cell marker protein or a nucleic acid encoding said protein selected from the group consisting of CD105, CD90, and CD44, wherein said cell also expresses at least one cardiac transcription factor protein selected from the group consisting of dHAND, Gata4, Mef2a, Tbx2, Tbx5, Tbx20, or an mRNA encoding said at least one cardiac transcription factor, and further wherein, said cell does not substantially express a c-kit protein or an mRNA encoding c-kit at the time of cell isolation.

40. The pharmaceutical composition of claim 39, wherein said cell further expresses an Abcg2 protein or an mRNA encoding Abcg2.

41. The pharmaceutical composition of claim 39, where said cell does not express at least one hematopoietic biomarker protein or a nucleic acid encoding said biomarker protein selected from the group consisting of CD45, CD34, CD31, Lin, Lmo2, Gata2, and Tal.

42. The pharmaceutical composition of claim 39, wherein said cell is capable of differentiating into a cardiac muscle cell.

43. A cultured human adult progenitor cell, wherein said cell is isolated from a cardiac tissue sample, wherein said cell expresses at least one mesenchymal stem cell marker protein or a nucleic acid encoding said protein selected from the group consisting of CD105, CD90, and CD44, wherein said cell also expresses at least one cardiac transcription factor protein selected from the group consisting of dHAND, Gata4, Mef2a, Tbx2, Tbx5, Tbx20, or an mRNA encoding said at least one cardiac transcription factor, and further wherein, said cell does not substantially express a c-kit protein or an mRNA encoding c-kit at the time of cell isolation.

44. The cell of claim 43, wherein said cell further expresses an Abcg2 protein or an mRNA encoding Abcg2.

45. The cell of claim 43, where said cell does not express at least one hematopoietic biomarker protein or a nucleic acid encoding said biomarker protein selected from the group consisting of CD45, CD34, CD31, Lin, Lmo2, Gata2, and Tal.

46. The cell of claim 43, wherein said cell is capable of differentiating into a cardiac muscle cell.

47. A pharmaceutical composition comprising a therapeutic amount of a cultured human adult progenitor cell, wherein said cell is isolated from a cardiac tissue sample, wherein said cell expresses at least one mesenchymal stem cell marker protein or a nucleic acid encoding said protein selected from the group consisting of CD105, CD90, and CD44, wherein said cell also expresses at least one cardiac transcription factor protein selected from the group consisting of dHAND, Gata4, Mef2a, Tbx2, Tbx5, Tbx20, or an mRNA encoding said at least one cardiac transcription factor, and further wherein, said cell does not substantially express a c-kit protein or an mRNA encoding c-kit at the time of cell isolation.

48. The pharmaceutical composition of claim 47, wherein said cell further expresses an Abcg2 protein or an mRNA encoding Abcg2.

49. The pharmaceutical composition of claim 47, where said cell does not express at least one hematopoietic biomarker protein or a nucleic acid encoding said biomarker protein selected from the group consisting of CD45, CD34, CD31, Lin, Lmo2, Gata2, and Tal.

50. The pharmaceutical composition of claim 47, wherein said cell is capable of differentiating into a cardiac muscle cell.

51. A substantially pure population of adult human progenitor cells, wherein said population is derived from a cardiac tissue sample obtained from a human, wherein said population of cells is selected by fluorescence or magnetic cell sorting, and wherein said population of cells expresses at least one mesenchymal stem cell marker protein or a nucleic acid encoding said protein selected from the list consisting of CD105, CD90, and CD44, wherein said population of cells also expresses at least one cardiac transcription factor protein selected from the group consisting of dHAND, Gata4, Mef2a, Tbx2, Tbx5, Tbx20, or an mRNA encoding said at least one cardiac transcription factor, and further wherein, said population of cells does not substantially express a c-kit protein or an mRNA encoding c-kit at the time of selection.

52. The population of adult human progenitor cells of claim 51, wherein said population of cells expresses an Abcg2 protein or an mRNA encoding Abcg2.

53. The population of adult human progenitor cells of claim 51, where said population of cells does not express at least one hematopoietic biomarker protein or a nucleic acid encoding said biomarker protein selected from the group consisting of CD45, CD34, CD31, Lin, Lmo2, Gata2, and Tal.

54. The population of adult human cardiac-derived progenitor cells of claim 51, wherein said population of cells is capable of differentiating into cardiac muscle cells.

55. A composition comprising a therapeutic amount of an isolated adult human progenitor cell, wherein said cell is derived from a cardiac tissue sample obtained from a human, wherein said cell expresses at least one mesenchymal stem cell marker protein or a nucleic acid encoding said protein selected from the group consisting of CD 105, CD90, and CD44, wherein said cell also expresses at least one cardiac transcription factor protein selected from the group consisting of dHAND, Gata4, Mef2a, Tbx2, Tbx5, Tbx20, or an mRNA encoding said at least one cardiac transcription factor, and further wherein, said cell does not substantially express a c-kit protein or an mRNA encoding c-kit at the time of cell isolation.

56. The composition of claim 55, wherein said composition further comprises a pharmaceutical acceptable carrier.

57. The composition of claim 55, wherein said cell further expresses an Abcg2 protein or an mRNA encoding Abcg2.

58. The composition of claim 55, where said cell does not express at least one hematopoietic biomarker protein or a nucleic acid encoding said biomarker protein selected from the group consisting of CD45, CD34, CD31, Lin, Lmo2, Gata2, and Tal.

59. The pharmaceutical composition of claim 55, wherein said cell is capable of differentiating into a cardiac muscle cell.

60. A method of treating a patient with heart disease, said method comprising the steps of administering to said patient a therapeutically effective amount of a human progenitor cell, wherein said cell is isolated from a cardiac tissue sample, wherein when said cell is isolated, said cell expresses at least one mesenchymal stem cell marker protein or a nucleic acid encoding said protein selected from the group consisting of CD105, CD90, and CD44, wherein said cell also expresses at least one cardiac transcription factor protein selected from the group consisting of dHAND, Gata4, Mef2a, Tbx2, Tbx5, Tbx20, or an mRNA encoding said at least one cardiac transcription factor, and further wherein, said cell does not substantially express a c-kit protein or an mRNA encoding c-kit at the time of cell isolation.

61. The method of claim 60, wherein said cell further expresses an Abcg2 protein or an mRNA encoding Abcg2.

62. The method of claim 60, where said cell does not express at least one hematopoietic biomarker protein or a nucleic acid encoding said biomarker protein selected from the group consisting of CD45, CD34, CD31, Lin, Lmo2, Gata2, and Tal.

63. The method of claim 60, wherein said cardiovascular disease is selected from the list consisting of coronary artery disease, myocardial infarction, ischemic heart disease, and heart failure.

64. The method of claim 60, wherein said cell is admixed with a pharmaceutical acceptable carrier.

65. The method of claim 60, wherein said cell is autologous.

66. A method of treating damaged myocardium in a patient, said method comprising the steps of:
(a) obtaining cells from an adult cardiac tissue sample;
(b) selecting cells one or more times, before or after expanding said cells, to generate a substantially pure population of cardiac progenitor cells that are c-kit^{neg} and positive for at least one mesenchymal stem cell marker selected from the list consisting of CD105, CD90, and CD44 at the time of cell selection;
(c) expanding said selected cells to form a substantially pure population of adult human cardiac progenitor cells;
(d) administering a therapeutically effective amount of said cardiac progenitor cells so expanded to said patient.

67. A method of obtaining a population of human cardiac progenitor cells, said method comprising the steps of:
(a) obtaining cells from a cardiac tissue sample;
(b) selecting cells one or more times, before or after expanding said cells, using either fluorescent or magnetic cell sorting techniques, to generate a substantially pure population of cardiac progenitor cells, wherein said cells are substantially c-kit^{neg} and positive for at least one mesenchymal stem cell marker selected from the list consisting of CD105, CD90, and CD44 at the time of sorting;
(c) expanding said selected cells to form a substantially pure population of adult human cardiac progenitor cells.

68. The method of claim 67, wherein the cells are sorted at least once using one or more biomarkers.

69. A method of treating damaged myocardium in a patient, said method comprising the steps of:
(a) obtaining cells from an adult cardiac tissue sample;
(b) selecting cells one or more times, before or after expanding said cells, to generate a substantially pure population of cardiac progenitor cells that are c-kit^{neg} and positive for at least one marker selected from the list consisting of Abcg2, CD 105, CD90, and CD44 at the time of cell selection;
(c) expanding said selected cells to form a substantially pure population of adult human cardiac progenitor cells;
(d) administering a therapeutically effective amount of said cardiac progenitor cells so expanded to said patient.

70. A method of obtaining a population of human cardiac progenitor cells, said method comprising the steps of:
(a) obtaining cells from a cardiac tissue sample;
(b) selecting cells one or more times, before or after expanding said cells, using either fluorescent or magnetic cell sorting techniques, to generate a substantially pure population of cardiac progenitor cells, wherein said cells are substantially c-kit^{neg} and positive for at least one marker selected from the list consisting of Abcg2, CD105, CD90, and CD44 at the time of sorting;
(c) expanding said selected cells to form a substantially pure population of adult human cardiac progenitor cells.

71. The method of claim 67, wherein the cells are sorted at least once using one or more biomarkers.
